SEQUENCE LISTING (3 sequences)

SEQ ID NO: 1 - protein, 93 aa, Homo sapiens
  MISC_FEATURE: C-C motif chemokine 14 Swiss-Prot Q16627
MKISVAAIPFFLLITIALGTKTESSSRGPYHPSECCFTYTTYKIPRQRIMDYYETNSQCSKPGIVFITKRGHSVCTNPSDKWVQDYIKDMKEN

SEQ ID NO: 2 - protein, 109 aa, Homo sapiens
  MISC_FEATURE: C-C motif chemokine 14 isoform 2
MKISVAAIPFFLLITIALGTKTESSSQTGGKPKVVKIQLKLVGGPYHPSECCFTYTTYKIPRQRIMDYYETNSQCSKPGIVFITKRGHSVCTNPSDKWVQDYIKDMKEN

SEQ ID NO: 3 - protein, 267 aa, Homo sapiens
  MISC_FEATURE: kallikrein-14 peptide Swiss-Prot Q9P0G3
MSLRVLGSGTWPSAPKMFLLLTALQVLAIAMTQSQEDENKIIGGHTCTRSSQPWQAALLAGPRRRFLCGGALLSGQWVITAAHCGRPILQVALGKHNLRRWEATQQVLRVVRQVTHPNYNSRTHDNDLMLLQLQQPARIGRAVRPIEVTQACASPGTSCRVSGWGTISSPIARYPASLQCVNINISPDEVCQKAYPRTITPGMVCAGVPQGGKDSCQGDSGGPLVCRGQLQGLVSWGMERCALPGYPGVYTNLCKYRSWIEETMRDK

(19) United States
(12) Patent Application Publication
(10) Pub. No.: US 2024/0027473 A1
(43) Pub. Date: Jan. 25, 2024

(54) METHODS AND COMPOSITIONS FOR TREATMENT OF RENAL INJURY AND RENAL FAILURE

(71) Applicant: ASTUTE MEDICAL, INC., SAN DIEGO, CA (US)

(72) Inventors: PAUL MCPHERSON, ENCINITAS, CA (US); JAMES PATRICK KAMPF, SAN DIEGO, CA (US); THOMAS KWAN, SAN DIEGO, CA (US)

(73) Assignee: ASTUTE MEDICAL, INC., SAN DIEGO, CA (US)

(21) Appl. No.: 18/253,293

(22) PCT Filed: Nov. 18, 2021
(86) PCT No.: PCT/US2021/059897

§ 371 Date: May 17, 2023

Related U.S. Application Data
(60) Provisional application No. 63/115,407, filed on Nov. 18, 2020.

(51) Int. Cl. G01N 33/68; G16H 20/40; A61K 35/14

(52) U.S. Cl. CPC G01N 33/6893; G16H 20/40; A61K 35/14; G01N 2800/52; G01N 2800/347

(57) ABSTRACT

A method of devising a therapy plan for renal replacement therapy (RRT) includes detecting a level of one or more biomarkers in a body fluid sample obtained from a subject. The level(s) may be correlated to an expected benefit from treatment with continued RRT and/or to an expected ability to successfully discontinue RRT. The method may include the step of assigning the subject to a predetermined subpopulation of individuals exhibiting a known status with regard to meeting criteria for continuing or discontinuing RRT. In some embodiments, the biomarker level is detected by introducing the body fluid into an assay instrument and contacting the body fluid to a binding reagent, for example, an antibody.

### CROSS REFERENCED APPLICATIONS

This application claims the benefit of U.S. Provisional Application No. 63/115,407 filed on Nov. 18, 2020, which is herein incorporated by reference in its entirety.

### SEQUENCE LISTING

This application contains a sequence listing which has been submitted in ASCII format via EFS-Web and is hereby incorporated by reference in its entirety. Said ASCII copy, created on Nov. 17, 2021, is A105893_1600 WO_SL_ST25.txt and is 4,714 bytes in size.

### BACKGROUND

The kidney is responsible for water and solute excretion from the body. Its functions include maintenance of acid-base balance, regulation of electrolyte concentrations, control of blood volume, and regulation of blood pressure. As such, loss of kidney function through injury and/or disease results in substantial morbidity and mortality. A detailed discussion of renal injuries is provided in Harrison's Principles of Internal Medicine, 17^{th} Ed., McGraw Hill, New York, pages 1741-1830, which is herein incorporated by reference in its entirety. Renal disease and/or injury may be acute or chronic. Acute and chronic kidney disease are described as follows (from Current Medical Diagnosis & Treatment 2008, 47^{th} Ed, McGraw Hill, New York, pages 785-815, which is herein incorporated by reference in its entirety): “Acute renal failure is worsening of renal function over hours to days, resulting in the retention of nitrogenous wastes (such as urea nitrogen) and creatinine in the blood. Retention of these substances is called azotemia. Chronic renal failure (chronic kidney disease) results from an abnormal loss of renal function over months to years”.

Acute Kidney Injury (AKI, also known as acute renal failure, or ARF) is an abrupt (typically detected within about 48 hours to 1 week) reduction in glomerular filtration. This loss of filtration capacity results in retention of nitrogenous (urea and creatinine) and non-nitrogenous waste products that are normally excreted by the kidney, a reduction in urine output, or both. It is reported that AKI complicates more than 10% of hospital admissions, 4-15% of cardiopulmonary bypass surgeries, and approaching up to two-thirds of intensive care admissions with survival being related, not only to the severity, but also to the duration of renal dysfunction. See, Hoste E A, Bagshaw S M, Bellomo R, Cely C M, Colman R, Cruz D N, et al. Intensive Care Med 2015; 41(8):1411-23; Mehta S, Chauhan K, Patel A, Patel S, Pinotti R, Nadkarni G N, et al., BMC Nephrology. 2018;19(1):91, each of which is herein incorporated by reference in its entirety. AKI is a major global cause of both morbidity and mortality. It is estimated that at least half of AKI cases resolve within 72 hours. Cases of AKI that resolve within 72 hours tend to have markedly better outcomes compared to cases which persist for at least 72 hours, especially for cases of severe AKI. Oliguria lasting at least 72 hours has been identified as a criterion for initiation renal replacement therapy (RRT). See, Gaudry S, Hajage D, Schortgen F, Martin-Lefevre L, Pons B, Boulet E, et al. The New England Journal of Medicine. 2016; 375(2):122-33, which is herein incorporated by reference in its entirety. Recent evidence suggests that two-thirds of patients with AKI resolve their renal dysfunction within 3-7 days whereas those who persist have dramatically reduced survival over the following year. See, Kellum J A, Sileanu F E, Bihorac A, Hoste E A, Chawla L S. Am J Respir Crit Care Med. 2017; 195(6):784-91, which is herein incorporated by reference in its entirety. Persistence of AKI at one week or more, termed acute kidney disease (AKD), is of grave importance in that it increases an individual's risk of developing chronic kidney disease and the consequences thereof. This link to chronic kidney disease (CKD) has been established over the last decade and specific recommendations for the management of patients with AKD have been proposed in order to try to influence this transition. See, Chawla L S, Bellomo R, Bihorac A, Goldstein S L, Siew E D, Bagshaw S M, et al., Nat Rev Nephrol. 2017; 13(4):241-57; Chawla L S, Eggers P W, Star R A, Kimmel P L, The New England Journal of Medicine 2014; 371(1):58-66, each of which is herein incorporated by reference in its entirety. It follows that early identification of individuals at risk of AKD would enable appropriate delivery of these proposed interventions, but also may identify individuals where newer therapies to attenuate AKI could be targeted.

Not only is persistence of AKI relevant to longer term outcomes, but clinical decision-making is also critically affected by physician expectations surrounding renal recovery and the decision of when to initiate RRT. Currently, this is almost totally dependent on clinical expectations as to the likelihood of recovery with no commercially available diagnostics to aid this decision process. As such, significant controversy exists around the timing of RRT with studies showing that some patients can benefit from the earlier initiation of RRT, while other studies demonstrate that some individuals receive RRT who may not require such treatment as they will recover renal function soon. See, Bagshaw S M, Lamontagne F, Joannidis M, Wald R. Critical care 2016;20(1):245; Forni to L G, Joannidis M. Nat Rev Nephrol 2019; 15(1):5-6, each of which is herein incorporated by reference in its entirety. It follows that early and reliable identification of those who will recover renal function may enable treatment to be stratified and avoid the incumbent risks of extracorporeal therapy. These risks include vascular access, hemodynamic instability, infection, as well as clearance of trace elements, water-soluble vitamins, and drugs. See, Ostermann M, Joannidis M, Pani A, Floris M, De Rosa S, Kellum J A, and Ronco C. Blood Purif. 2016; 42(3):224-37, which is herein incorporated by reference in its entirety. Other considerations include the availability of machines and clinical staff, healthcare costs, and patient immobility. Id.

Furthermore, even less is known about the optimal conditions under which to discontinue RRT after initiation. Both premature and delayed discontinuation of RRT can have negative impacts on patient outcomes and resource utilization. See, Katulka R J, Al Saadon A, Sebastianski M, Featherstone R, Vandermeer B, Silver S A, et al. Crit Care 2020; 24(1):50; Kelly Y P, Waikar S S, Mendu M L. Semin Dial 2019, 32(3):205-209, each of which is herein incorporated by reference in its entirety.

These challenges underscore the need for better methods to detect and assess which AKI patients will benefit from treatment with continued RRT and distinguish them from those who will improve without treatment with continued RRT or who will fail to improve despite treatment with continued RRT.

### SUMMARY

Methods and compositions for assessing a likelihood that a subject will benefit from treatment with continued renal replacement therapy (RRT) are provided. As described herein, measurement of C-C motif chemokine 14 (CCL14) may be used as a biomarker to indicate whether a subject will benefit from treatment with continued RRT. Alternatively, or in combination with CCL14, kallikrein-14 (KLK14) may be used as a biomarker to indicate whether a subject will benefit from treatment with continued RRT. Furthermore, the following biomarkers, alone or in combination, may be used as a biomarker(s) to indicate whether a subject will benefit from treatment with continued RRT: C-C motif chemokine 14, kallikrein-14, antileukoproteinase, cathepsin B, C-C motif chemokine 1, C-C motif chemokine 16, C-C motif chemokine 23, C-C motif chemokine 24, C-C motif chemokine 28, chitinase-3-like protein 1, C-X-C motif chemokine 2, C-X-C motif chemokine 9, dickkopf-related protein 1, elafin, fatty acid-binding protein adipocyte, follistatin-related protein 3, hepatocyte growth factor-like protein, insulin-like growth factor-binding protein 2, insulin-like growth factor binding protein 4, insulin-like growth factor binding protein 7, metalloproteinase inhibitor 1, metalloproteinase inhibitor 2, metalloproteinase inhibitor 4, neutrophil gelatinase-associated lipocalin, nidogen-1, OX-2 membrane glycoprotein, pro-interleukin-16, prolactin, renin, tissue factor pathway inhibitor, tumor necrosis factor receptor superfamily member 10B, tumor necrosis factor receptor superfamily member 18, tumor necrosis factor receptor superfamily member 6B, and WNT1-inducible signaling pathway protein 1.

The benefit to the subject of treatment with continued RRT may include one or more of increased renal function, increased glomerular filtration rate (GFR), reduced serum creatinine, increased urine output, hemodynamic optimization, regulated blood pressure, regulated electrolyte and vitamin levels, increased life expectancy, and increased standard of living. This list is not meant to be limiting.

In one aspect of the disclosure, a method for assessing the likelihood that a subject will benefit from treatment with continued RRT comprises detecting by an analyte binding assay a level of one or more of the aforementioned biomarkers in at least one body fluid sample to produce one or more assay results. The one or more assay results may be used individually or in combination with each other (e.g., two or more assay results may be combined into a single composite assay result). In some embodiments, the one or more biomarkers may comprise or may consist of CCL14. In some embodiments, the one or more biomarkers may comprise or may consist of KLK14. In some embodiments, the one or more biomarkers may comprise or may consist of CCL14 and KLK14. The method further comprises correlating the one or more assay results to a likelihood that the subject will benefit from treatment with continued RRT or a likelihood that the subject will not benefit from treatment with continued RRT. The subject may be receiving RRT at the time the sample is obtained. The method may include the step of determining a duration of prospective RRT treatment. The method may include the step of treating the subject, according to the results of the correlation step. For example, the subject may be treated by either continuing or discontinuing RRT. The treatment may be based on the subject's likelihood of benefiting from treatment with continued RRT. The treatment may comprise administering continued RRT if the subject is at an increased likelihood of benefiting from continued RRT. The treatment may comprise discontinuing an RRT treatment if the subject is not an increased likelihood of benefiting from treatment with continued RRT (i.e. an increased likelihood that the subject will not benefit from administering continued RRT). In some embodiments in which the treatment comprises continued RRT, the RRT may be administered for at least about 6, 8, 12, 24, 48, or 72 hours following when the one or more body fluid sample was collected. In some embodiments in which the treatment comprises discontinuing RRT, RRT may be discontinued immediately (substantially concurrent with receipt of test results) and/or within about 6, 8, 12, 24, 48, or 72 hours of when the one or more body fluid sample was collected.

A variety of types of RRT are available which may include different dialysis line insertion sites and different protocols with regard to timing dialysis sessions. The step of treating a subject with continued RRT may comprise one or more of the following types of dialysis: continuous renal replacement therapy, intermittent renal replacement therapy, prolonged intermittent renal replacement therapy, continuous hemodialysis, continuous hemofiltration, continuous hemodiafiltration, intermittent hemodialysis, intermittent hemofiltration, intermittent hemodiafiltration, acute hemodialysis, peritoneal dialysis, slow continuous ultrafiltration, and sustained low efficiency dialysis. See, e.g., Gemmel L et al., BJA Education 2017, 17(3): 88-93; Claure-Del Granado R, “Role of Acute Dialysis (CRRT, SLED, Intermittent hemodialysis, other),” Renal & Urology News 2017, each of which are herein incorporated by reference in their entirety. In addition to dialysis, RRT may comprise renal transplantation. Discontinuing RRT may comprise discontinuing one of the aforementioned types of dialysis.

The correlation step may comprise the step of assigning the subject to a predetermined subpopulation of individuals exhibiting a known status with regard to benefiting from administration of continued RRT. The assignment may be made by comparing each of the one or more assay results to an individual threshold for a specific biomarker selected in a population study. Where a plurality of assay results are used, the assignment may be made by comparing a singular composite of two or more of the assay results to a respective composite threshold. The threshold used may separate the population into a first subpopulation above the threshold which has an increased predisposition for benefiting from treatment with continued RRT relative to a second subpopulation at or below the threshold. In some instances, the predisposition may be to benefit from more than 1, more than 2, or more than 3 days of prospective treatment with RRT (e.g., at least 2, at least 3, or at least 4 days). The threshold may separate the population into a second subpopulation at or below the threshold which has a decreased predisposition for benefiting from treatment with continued RRT relative to the first subpopulation above the threshold. In some instances, the predisposition may be to benefit from more than 1, more than 2, or more than 3 days of prospective treatment with RRT (e.g., at least 2, at least 3, or at least 4 days).

The correlation step may comprise the step of assigning the subject to a predetermined subpopulation of individuals exhibiting a known status with regard to having or for not having successfully discontinued RRT. The assignment may be made by comparing each of the one or more assay results to an individual threshold selected in a population study. Where a plurality of assay results are used, the assignment may be made by comparing a singular composite of two or more of the assay results to a respective composite threshold. The threshold used may separate the population into a first subpopulation having measurements above the threshold which has not successfully discontinued RRT and a second population having measurements at or below the threshold which has successfully discontinued RRT. In some instances, the successful discontinuation of RRT may have been within 1, 2, or 3 days from the time at which the measurements were made.

The correlation step may comprise comparing an assay result (e.g., an individual assay result or composite assay result) to a baseline previously measured in the subject. The baseline may have been measured during a time the subject was not on RRT.

In some implementations, the subject may be assigned to one of the first subpopulations described above and/or an assay result may be higher than a baseline described above. Such a subject may be treated by administering continued RRT (e.g., continuing RRT). The RRT may be administered for more than 1, more than 2, or more than 3 days following sampling (e.g., for at least 2, at least 3, or at least 4 days).

In some implementations, the subject may be assigned to one of the second subpopulations described above and/or an assay result may not be higher than a baseline described above. Such a subject may be treated by discontinuing an RRT treatment. In some instances, RRT may be discontinued immediately (substantially concurrent with the receipt of test results). In some instances, the RRT may be discontinued within 1, 2, or 3 days of the time at which the sample is obtained. Alternatively, in some instances, the RRT may be maintained for 1, 2, or 3 days before the RRT is subsequently discontinued.

In some embodiments, the analyte binding assay may comprise an antibody. The at least one body fluid sample may comprise a urine sample, a whole blood sample, a plasma sample, or a serum sample. In some embodiments, the at least one body fluid sample comprises both a urine sample and a plasma sample.

In some embodiments, the subject may present with pathologies in addition to AKI or other renal disease/dysfunction for which the subject is receiving RRT. For example, the subject may have (e.g., have been diagnosed with or suspected as potentially having) one or more of the following pathologies: congestive heart failure, diabetes mellitus, hypertension, coronary artery disease, proteinuria, cirrhosis, chronic kidney disease, cancer, chronic obstructive pulmonary disease, anemia, sepsis, shock, and hypotension. The subject may have experienced surgery or trauma within about 12, 24, 36, 48, 72, 96, or 120 hours prior to the time the sample is obtained. The sample may have been collected within about 6, 8, 12, 24, 36, 48, or 72 hours of RRT having been initiated. The subject may have acute kidney injury (AKI) at the time the sample was obtained. The subject may be in KDIGO stage 2 or 3 acute kidney injury at the time the sample was obtained. RRT may have been initiated within about 6, 8, 12, 24, 36, 48, or 72 hours of the subject meeting the criteria for KDIGO stage 2 acute kidney injury. RRT may have been initiated within about 6, 8, 12, 24, 36, 48, or 72 hours of the subject meeting the criteria for KDIGO stage 3 acute kidney injury.

The biomarker levels may be measured in one or more body fluid samples obtained from the subject. In some embodiments, this is accomplished by introducing a single body fluid sample selected from the one or more collected body fluid samples into an assay instrument. The measurement may be produced by an analyte binding assay performed by the analyte assay instrument. The assay instrument may cause all or a portion of the body fluid sample to come in contact with one or more binding reagents. Each of the one or more binding reagents may bind a single specific biomarker in the body fluid sample for detection of the biomarker. For example, a binding agent may bind C-C motif chemokine 14, kallikrein-14, or any of the other biomarkers listed above. The assay instrument may then generate for each of the specific biomarkers one or more assay results which indicate binding of the specific biomarker to the binding reagent.

In some embodiments, the binding reagent within or associated with the assay instrument may include an antibody. In some embodiments, the antibody may be a monoclonal antibody. In some embodiments, the binding reagent may include a fragment of an antibody. In some embodiments, for each of the specific biomarkers for which the assay instrument generates an assay result, it may do so by contacting the bound biomarker with a secondary binding reagent which also binds the biomarker. The secondary binding reagent may be conjugated to a detectable label for producing a detectable signal. The detectable label may be different for each of the specific biomarkers. In some embodiments, the secondary binding reagent may include an antibody. In some embodiments, the antibody may be a monoclonal antibody. In some embodiments, the secondary binding reagent may include a fragment of an antibody. The one or more binding reagents may be a plurality of binding reagents, each being specific to a different biomarker. Each binding reagent of the plurality of binding reagents may be bound to a different zone of the assay instrument.

In some embodiments, the subject may present with one or more of the following clinical indications upon performing the aforementioned assay(s): (i) oliguria or anuria for more than 72 hours, (ii) blood urea nitrogen of more than 40 mmol/L, (iii) serum potassium concentration of more than 6 mmol/L, (iv) serum potassium concentration of more than 5.5 mmol/L despite treatment with bicarbonate, glucose-insulin infusion, or both, (v) pH below 7.15 in a context of pure metabolic acidosis or in a context of mixed acidosis, and (vi) acute pulmonary edema. In some embodiments, the subject may present with one or more of the following indications for RRT: volume overload, acid-base abnormalities (e.g., severe metabolic acidosis), electrolyte abnormalities (e.g., severe hyperkalemia, severe hyponatremia, severe hypernatremia, severe hyperphosphatemia, etc.), and overt uremic symptoms (e.g., encephalopathy, pericarditis, platelet dysfunction, impaired nutrition, heart failure, pulmonary edema, etc.). In instances of hyperkalemia, the subject may have serum potassium levels of at least about 6.0-6.5 mmol/L. Subjects may have any of the indications for continuous RRT generally described in Tandukar et al., Chest. 2019 Mar;155(3):626-638 (doi: 10.1016/j.chest.2018.09.004), which is herein incorporated by reference in its entirety.

In another aspect of the disclosure, a method of detecting one or more kidney injury markers in a subject comprises detecting a level of one or more of the aforementioned biomarkers in a body fluid sample obtained from the subject. In some embodiments, the one or more biomarkers comprises or consists of C-C motif chemokine 14. In some embodiments, the one or more biomarkers comprises or consists of kallikrein-14. In some embodiments, the one or more biomarkers comprises or consists of C-C motif chemokine 14 and kallikrein-14. The subject may have (e.g., have been diagnosed with or suspected as potentially having) injury to renal function, reduced renal function, acute kidney injury, persistent acute kidney injury, acute kidney disease, or chronic kidney disease. The subject may have been receiving renal replacement therapy (RRT) at the time the sample was obtained. The method may include obtaining the sample from the subject. The body fluid sample may be urine, whole blood, serum, or plasma. The sample may be obtained from a subject described in the methods described above or elsewhere herein.

The disclosure includes a kit which comprises components which may be used to perform one or more of the disclosed methods. The kit may comprise one or more binding reagents, each of which may bind C-C motif chemokine 14, kallikrein-14, and/or any of the biomarkers disclosed herein. The kit may comprise at least two binding reagents (to bind at least two of the one or more biomarkers). One or more of binding reagents in the kit may comprise an antibody. In an example, the antibody is a monoclonal antibody. In another example, the antibody is a fragment of an antibody.

In another aspect of the disclosure, disclosed herein is a system which may comprise one of the foregoing kits. The system further comprises an assay instrument configured to receive one or more body fluid samples and generate an assay result corresponding to a level of a biomarker in the body fluid sample for each marker detected by the reagents of the kit.

### DETAILED DESCRIPTION

Disclosed herein are methods and compositions for determination of appropriate treatment regimens in subjects receiving RRT and/or for determining the duration of prospective treatment with RRT in a subject. Subjects may be suffering from acute kidney injury (AKI), acute kidney disease (AKD), chronic kidney disease (CKD) or other renal disease or renal dysfunction. These treatments regimens may comprise renal replacement therapy (RRT). In various aspects, a measured concentration or level of one or more biomarkers, for example, C-C motif chemokine 14 (CCL14), kallikrein-14 (KLK14), and/or one or more additional kidney injury markers disclosed herein or known in the art, are correlated to a designation that the subject is or is not a suitable candidate for treatment with continued RRT. The correlation may be used to guide therapy for the subject with regard to administering continued RRT (e.g., continuing or discontinuing RRT).

As used herein, “continued” RRT refers to RRT of some extended duration of time (e.g., prospective RRT continued for more than 1 day, 2 days, 3 days, etc.) and “continuing RRT” refers to keeping a subject on RRT for at least some extended duration of time. “Discontinuing RRT” refers to removing a subject from RRT for at least some extended duration of time (e.g., for at least 1 day, 2 days, 3 days, etc.). Discontinuation of RRT may also be referred to as liberation from RRT, cessation of RRT, or RRT weaning, as is understood in the field. See, e.g., Katulka R J, Al Saadon A, Sebastianski M, Featherstone R, Vandermeer B, Silver S A, et al. Crit Care 2020; 24(1):50, which is herein incorporated by reference in its entirety. As will be understood herein, if a subject is treated with continued RRT then the subject has not been treated with discontinuation of RRT, and if a subject is treated with discontinuation of RRT then the subject has not been treated with continued RRT. The subject may be presently on RRT when tested (i.e. when one or more biomarker levels according to the present disclosure are measured) to determine whether the subject would likely benefit from continued RRT or not (e.g., would benefit from discontinuing RRT).

As used herein, an “injury to renal function” is an abrupt (within 14 days, preferably within 7 days, more preferably within 72 hours, and still more preferably within 48 hours) measurable reduction in a measure of renal function. Such an injury may be identified, for example, by a decrease in glomerular filtration rate (GFR) or estimated GFR, a reduction in urine output, an increase in serum creatinine, an increase in serum cystatin C, a requirement for renal replacement therapy, etc. “Improvement in Renal Function” is an abrupt (within 14 days, preferably within 7 days, more preferably within 72 hours, and still more preferably within 48 hours) measurable increase in a measure of renal function.

As used herein, “reduced renal function” is an abrupt (within 14 days, preferably within 7 days, more preferably within 72 hours, and still more preferably within 48 hours) reduction in kidney function identified by an absolute increase in serum creatinine of greater than or equal to 0.1 mg/dL (≥8.8 μmol/L), a percentage increase in serum creatinine of greater than or equal to 20% (1.2-fold from baseline), or a reduction in urine output (documented oliguria of less than 0.5 ml/kg per hour).

As used herein, “acute kidney injury” or “AKI” is an abrupt (within 14 days, preferably within 7 days, more preferably within 72 hours, and still more preferably within 48 hours) reduction in kidney function identified by an absolute increase in serum creatinine of greater than or equal to 0.3 mg/dl (≥26.4 μmol/I), a percentage increase in serum creatinine of greater than or equal to 50% (1.5-fold from baseline), or a reduction in urine output (documented oliguria of less than 0.5 ml/kg per hour for at least 6 hours). This term is synonymous with “acute renal failure” or “ARF.”

AKI may be categorized as prerenal, intrinsic renal, or postrenal in causation. Intrinsic renal disease can be further divided into glomerular, tubular, interstitial, and vascular abnormalities. Major causes of AKI are described in the following table, which is adapted from the Merck Manual, 17^{th} ed., Chapter 222, and which is hereby incorporated by reference in its entirety:

| Type | Risk Factors |
|---|---|
| Prerenal | |
| ECF volume | Excessive diuresis, hemorrhage, GI losses, loss |
| depletion | of intravascular fluid into the extravascular space |
| | (due to ascites, peritonitis, pancreatitis, or burns), |
| | loss of skin and mucus membranes, renal salt- |
| | and water-wasting states |
| Low cardiac output | Cardiomyopathy, MI, cardiac tamponade, |
| | pulmonary embolism, pulmonary hypertension, |
| | positive-pressure mechanical ventilation |
| Low systemic | Septic shock, liver failure, antihypertensive drugs |
| vascular resistance | |
| Increased renal | NSAIDs, cyclosporines, tacrolimus, |
| vascular resistance | hypercalcemia, anaphylaxis, anesthetics, renal |
| | artery obstruction, renal vein thrombosis, sepsis, |
| | hepatorenal syndrome |
| Decreased efferent | ACE inhibitors or angiotensin II receptor blockers |
| arteriolar tone | |
| (leading to decreased | |
| GFR from reduced | |
| glomerular | |
| transcapillary | |
| pressure, especially | |
| in patients with | |
| bilateral renal artery | |
| stenosis) | |
| Intrinsic Renal | |
| Acute tubular injury | Ischemia (prolonged or severe prerenal state): |
| | surgery, hemorrhage, arterial or venous |
| | obstruction; Toxins: NSAIDs, cyclosporines, |
| | tacrolimus, aminoglycosides, foscarnet, ethylene |
| | glycol, hemoglobin, myoglobin, ifosfamide, heavy |
| | metals, methotrexate, radiopaque contrast |
| | agents, streptozotocin |
| Acute | ANCA-associated: Crescentic |
| glomerulonephritis | glomerulonephritis, polyarteritis nodosa, |
| | Wegener's granulomatosis; Anti-GBM |
| | glomerulonephritis: Goodpasture's syndrome; |
| | Immune-complex: Lupus glomerulonephritis, |
| | postinfectious glomerulonephritis, |
| | cryoglobulinemic glomerulonephritis |
| Acute | Drug reaction (eg, β-lactams, NSAIDs, |
| tubulointerstitial | sulfonamides, ciprofloxacin, thiazide diuretics, |
| nephritis | furosemide, phenytoin, allopurinol, |
| | pyelonephritis, papillary necrosis |
| Acute vascular | Vasculitis, malignant hypertension, thrombotic |
| nephropathy | microangiopathies, scleroderma, |
| | atheroembolism |
| Infiltrative diseases | Lymphoma, sarcoidosis, leukemia |
| Postrenal | |
| Tubular precipitation | Uric acid (tumor lysis), sulfonamides, |
| | triamterene, acyclovir, indinavir, methotrexate, |
| | ethylene glycol ingestion, myeloma protein, |
| | myoglobin |
| Ureteral obstruction | Intrinsic: Calculi, clots, sloughed renal tissue, |
| | fungus ball, edema, malignancy, congenital |
| | defects; Extrinsic: Malignancy, retroperitoneal |
| | fibrosis, ureteral trauma during surgery or high |
| | impact injury |
| Bladder obstruction | Mechanical: Benign prostatic hyperplasia, |
| | prostate cancer, bladder cancer, urethral |
| | strictures, phimosis, paraphimosis, urethral |
| | valves, obstructed indwelling urinary catheter; |
| | Neurogenic: Anticholinergic drugs, upper or lower |
| | motor neuron lesion |

In the case of ischemic AKI, the course of the disease may be divided into four phases. During an initiation phase, which lasts hours to days, reduced perfusion of the kidney is evolving into injury. Glomerular ultrafiltration reduces, the flow of filtrate is reduced due to debris within the tubules, and back leakage of filtrate through injured epithelium occurs. Renal injury can be mediated during this phase by reperfusion of the kidney. Initiation is followed by an extension phase which is characterized by continued ischemic injury and inflammation and may involve endothelial damage and vascular congestion. During the maintenance phase, lasting from 1 to 2 weeks, renal cell injury occurs, and glomerular filtration and urine output reaches a minimum. A recovery phase can follow in which the renal epithelium is repaired and GFR gradually recovers. Despite this, the survival rate of subjects with AKI may be as low as about 60%.

AKI may be caused by radiocontrast agents (also called contrast media) and other nephrotoxins such as cyclosporine, antibiotics including aminoglycosides and anticancer drugs such as cisplatin typically manifests over a period of days to—about a week. Contrast induced nephropathy (CIN, which is AKI caused by radiocontrast agents) is thought to be caused by intrarenal vasoconstriction (leading to ischemic injury) and from the generation of reactive oxygen species that are directly toxic to renal tubular epithelial cells. CIN classically presents as an acute (onset within 24-48 h) but reversible (peak 3-5 days, resolution within 1 week) rise in blood urea nitrogen and serum creatinine.

A commonly reported criterion for defining and detecting AKI is an abrupt (typically within about 2-7 days or within a period of hospitalization) elevation of serum creatinine. Although the use of serum creatinine elevation to define and detect AKI is well established, the magnitude of the serum creatinine elevation and the time over which it is measured to define AKI varies considerably among publications. Traditionally, relatively large increases in serum creatinine such as 100%, 200%, an increase of at least 100% to a value over 2 mg/dL and other definitions were used to define AKI. However, the recent trend has been towards using smaller serum creatinine rises to define AKI. The relationship between serum creatinine rise, AKI and the associated health risks are reviewed in Praught and Shlipak, Curr Opin Nephrol Hypertens 14:265-270, 2005 and Chertow et al, J Am Soc Nephrol 16: 3365-3370, 2005, which, with the references listed therein, are hereby incorporated by reference in their entirety. As described in these publications, acute worsening renal function (AKI) and increased risk of death and other detrimental outcomes are now known to be associated with very small increases in serum creatinine. These increases may be determined as a relative (percent) value or a nominal value. Relative increases in serum creatinine as small as 20% from the pre-injury value have been reported to indicate acutely worsening renal function (AKI) and increased health risk, but the more commonly reported value to define AKI and increased health risk is a relative increase of at least 25%. Nominal increases as small as 0.3 mg/dL, 0.2 mg/dL or even 0.1 mg/dL have been reported to indicate worsening renal function and increased risk of death. Various time periods for the serum creatinine to rise to these threshold values have been used to define AKI, for example, ranging from 2 days, 3 days, 7 days, or a variable period defined as the time the patient is in the hospital or intensive care unit. These studies indicate there is not a particular threshold serum creatinine rise (or time period for the rise) for worsening renal function or AKI, but rather a continuous increase in risk with increasing magnitude of serum creatinine rise.

One study (Lassnigg et al, J Am Soc Nephrol 15:1597-1605, 2004) investigated both increases and decreases in serum creatinine. Patients with a mild fall in serum creatinine of −0.1 to −0.3 mg/dL following heart surgery had the lowest mortality rate. Patients with a larger fall in serum creatinine (more than or equal to −0.4 mg/dL) or any increase in serum creatinine had a larger mortality rate. These findings caused the authors to conclude that even very subtle changes in renal function (as detected by small creatinine changes within 48 hours of surgery) seriously effect patient's outcomes. In an effort to reach consensus on a unified classification system for using serum creatinine to define AKI in clinical trials and in clinical practice, Bellomo et al., Crit Care. 8(4):R204-12, 2004, which is hereby incorporated by reference in its entirety for the RIFLE criteria, proposes the following classifications for stratifying AKI patients:
“Risk”: serum creatinine increased 1.5 fold from baseline OR urine production of <0.5 ml/kg body weight/hr for 6 hours;
“Injury”: serum creatinine increased 2.0 fold from baseline OR urine production <0.5 ml/kg/hr for 12 h;
“Failure”: serum creatinine increased 3.0 fold from baseline OR creatinine 4.0 mg/dL (355 μmol/I) with an acute rise of 0.5 mg/dl (44 μmol/I) OR urine output below 0.3 ml/kg/hr for 24 h OR anuria for at least 12 hours; And included two clinical outcomes:
“Loss”: persistent need for renal replacement therapy for more than four weeks.
“ESRD”: end stage renal disease—the need for dialysis for more than 3 months.

These criteria are called the RIFLE criteria, which provide a useful clinical tool to classify renal status. As discussed in Kellum, Crit Care Med. 36: S141-45, 2008 and Ricci et al., Kidney Int. 73, 538-546, 2008, each hereby incorporated by reference in its entirety, the RIFLE criteria provide a uniform definition of AKI which has been validated in numerous studies. As will be understood in the art, RIFLE stage 0 can be used to classify a subject who does not meet the criteria for RIFLE stage R or any more severe RIFLE stage of AKI (i.e. a subject who does not have kidney injury or a subject who has a kidney injury but has not progressed to meeting any of the threshold criteria for RIFLE stage R or more severe RIFLE stages of AKI).

More recently, Mehta et al., Crit Care 11:R31 (doi:10.1186.cc5713), 2007, hereby incorporated by reference in its entirety, proposes the following similar classifications for stratifying AKI patients (AKIN), which have been modified from RIFLE:
“Stage I”: increase in serum creatinine of more than or equal to 0.3 mg/dL (≥26.4 μmol/L) or increase to more than or equal to 150% (1.5-fold) from baseline OR urine output less than 0.5 m L/kg per hour for more than 6 hours;
“Stage II”: increase in serum creatinine to more than 200% (>2-fold) from baseline OR urine output less than 0.5 mL/kg per hour for more than 12 hours;
“Stage III”: increase in serum creatinine to more than 300% (>3-fold) from baseline OR serum creatinine 4.0 mg/dL 354 μmol/L) accompanied by an acute increase of at least 0.5 mg/dL (44 μmol/L) OR urine output less than 0.3 m L/kg per hour for 24 hours or anuria for 12 hours.

As will be understood in the art, AKIN stage 0 can be used to classify a subject who does not meet the criteria for AKIN stage I or any more severe AKIN stage of AKI (i.e. a subject who does not have kidney injury or a subject who has a kidney injury but has not progressed to meeting any of the threshold criteria for AKIN stage I or more severe AKIN stages of AKI).

Likewise, Kidney Disease: Improving Global Outcomes (KDIGO) Acute Kidney Injury Work Group. KDIGO Clinical Practice Guideline for Acute Kidney Injury, Kidney inter., Suppl. 2012; 2: 1-138, refers to both RIFLE and AKIN, and offers the following AKI staging guidelines:

| Stage | Serum creatinine | or | Urine output |
|---|---|---|---|
| 1 | 1.5-1.9 times baseline | | <0.5 ml/kg/h for 6-12 hours |
| | or | | |
| | ≥0.3 mg/dl (≥26.5 mmol/l) | | |
| | increase | | |
| 2 | 2.0-2.9 times baseline | | <0.5 ml/kg/h for ≥12 hours |
| 3 | 3.0 times baseline | | <0.3 ml/kg/h for ≥24 hours |
| | or | | or |
| | Increase in serum creatinine to | | Anuria for ≥12 hours |
| | ≥4.0 mg/dl (≥353.6 mmol/l) | | |
| | or | | |
| | Initiation of renal replacement | | |
| | therapy | | |
| | or | | |
| | In patients <18 years, decrease in | | |
| | eGFR to <35 ml/min per 1.73 m² | | |

As will be understood in the art, KDIGO stage 0 can be used to classify a subject who does not meet the criteria for KDIGO stage 1 or any more severe KDIGO stage of AKI (i.e. a subject who does not have kidney injury or a subject who has a kidney injury but has not progressed to meeting any of the threshold criteria for KDIGO stage 1 or more severe KDIGO stages of AKI).

The CIN Consensus Working Panel (McCollough et al, Rev Cardiovasc Med. 2006; 7(4):177-197, hereby incorporated by reference in its entirety) uses a serum creatinine rise of 25% to define Contrast induced nephropathy (which is a type of AKI). Although various groups propose slightly different criteria for using serum creatinine to detect AKI, the consensus is that small changes in serum creatinine, such as 0.3 mg/dL or 25%, are sufficient to detect AKI (worsening renal function) and that the magnitude of the serum creatinine change is an indicator of the severity of the AKI and mortality risk.

These classification systems of AKI generally comprise serum creatinine criteria and urine output criteria for each stage. Wherever specified herein, any stage of AKI may be considered equivalent to (i.e. substituted with) any of the individual criteria that qualifies a subject as being at that particular stage of AKI. In some embodiments, the methods disclosed herein may also be used to correlate to a renal status defined by a particular AKI stage (e.g., the likelihood of reaching a particular AKI stage or the likelihood of persistent AKI at a particular stage), wherein the particular AKI stage can be defined by meeting both a serum creatinine criterion that qualifies the subject for that particular stage and a urine output criterion that qualifies a subject for that particular stage. In some embodiments, the particular AKI stage can be defined by meeting all the criteria (i.e. both of all the serum creatinine criteria and all the urine output criteria). All the methods disclosed herein may define stages of AKI according to any of these embodiments, unless stated otherwise. It will be understood in the art, that similarly defined stages of AKI may generally be interchanged with one another as relates to use of the biomarkers disclosed herein, unless dictated otherwise by context. That is, RIFLE stage R, AKIN stage I, and KDIGO stage 1 may generally be interchangeable; RIFLE stage I, AKIN stage II, and KDIGO stage 2 may generally be interchangeable; and RIFLE stage F, AKIN stage III, and KDIGO stage 3 may generally be interchangeable.

Although serial measurement of serum creatinine over a period of days is an accepted method of detecting and diagnosing AKI and is considered one of the most important tools to evaluate AKI patients, serum creatinine is generally regarded to have several limitations in the diagnosis, assessment and monitoring of AKI patients. The time period for serum creatinine to rise to values (e.g., a 0.3 mg/dL or 25% rise) considered diagnostic for AKI can be 48 hours or longer depending on the definition used. Since cellular injury in AKI can occur over a period of hours, serum creatinine elevations detected at 48 hours or longer can be a late indicator of injury, and relying on serum creatinine can thus delay diagnosis of AKI. Furthermore, serum creatinine is not a good indicator of the exact kidney status and treatment needs during the most acute phases of AKI when kidney function is changing rapidly. Some patients with AKI will recover fully, some will need dialysis (either short term or long term) and some will have other detrimental outcomes including death, major adverse cardiac events and chronic kidney disease. Because serum creatinine is a marker of filtration rate, it does not differentiate between the causes of AKI (pre-renal, intrinsic renal, post-renal obstruction, atheroembolic, etc.) or the category or location of injury in intrinsic renal disease (for example, tubular, glomerular or interstitial in origin). Urine output is similarly limited. Knowing these things can be of vital importance in managing and treating patients with AKI.

As used herein, “persistent AKI” refers to episodes of AKI that persist for at least 48-72 hours before sustained reversal. Reversal of AKI must generally last for a minimum of 48 hours to consider any subsequent episodes of AKI a distinct episode rather than persistence of the original episode. Definitions for various stages of renal injury, including persistent AKI, as well as methods for assessment and treatment may be found in Nat Rev Nephrol. 2017 April; 13(4):241-257 (doi: 10.1038/nrneph.2017.2), which is herein incorporated by reference in its entirety. Persistence of specific stages of AKI (e.g., KDIGO stage 3 AKI) may be defined in a similar manner, wherein a minimum stage of AKI must be maintained for 48-72 hours before sustained recovery from that stage.

As used herein, the term “C-C motif chemokine 14” refers to one or more polypeptides present in a biological sample that are derived from the C-C motif chemokine 14 precursor (human sequence: Swiss-Prot Q16627 (SEQ ID NO: 1)):

| | |
|---|---|
| MKISVAAIPF FLLITIALGT KTESSSRGPY HPSECCFTYT TYKIPRQRIM | 50 |
| DYYETNSQCS KPGIVFITKR GHSVCTNPSD KWVQDYIKDM KEN | 93 |

The following domains have been identified in C-C motif chemokine 14:

| Residues | Length | Domain ID |
|---|---|---|
| 1-19 | 19 | Signal peptide |
| 20-93 | 74 | C-C motif chemokine 14 |
| 22-93 | 72 | HCC-1(3-74) |
| 23-93 | 71 | HCC-1(4-74) |
| 28-93 | 66 | HCC-1(9-74) |
| 27 | | R→QTGGKPKVVKIQLKLVG in isoform 2 (SEQ ID NO: 2) |

As used herein, the term “kallikrein-14” refers to one or more polypeptides present in a biological sample that are derived from the kallikrein-14 peptide (human sequence: Swiss-Prot Q9P0G3 (SEQ ID NO: 3)):

| | |
|---|---|
| MSLRVLGSGT WPSAPKMFLL LTALQVLAIA MTQSQEDENK IIGGHTCTRS | 50 |
| SQPWQAALLA GPRRRFLCGG ALLSGQWVIT AAHCGRPILQ VALGKHNLRR | 100 |
| WEATQQVLRV VRQVTHPNYN SRTHDNDLML LQLQQPARIG RAVRPIEVTQ | 150 |
| ACASPGTSCR VSGWGTISSP IARYPASLQC VNINISPDEV CQKAYPRTIT | 200 |
| PGMVCAGVPQ GGKDSCQGDS GGPLVCRGQL QGLVSWGMER CALPGYPGVY | 250 |
| TNLCKYRSWI EETMRDK | 267 |

The following domains have been identified in kallikrein-14:

| Residues | Length | Domain ID |
|---|---|---|
| 1-34 | 34 | Signal peptide |
| 35-40 | 6 | Propeptide |
| 41-267 | 227 | Active enzyme |

As used herein, the term “relating a signal to the presence or amount” of an analyte reflects this understanding. Assay signals are typically related to the presence or amount (e.g., a concentration) of an analyte through the use of a standard curve calculated using known concentrations of the analyte of interest. As the term is used herein, an assay is “configured to detect” an analyte if an assay can generate a detectable signal indicative of the presence of or a level/amount of an analyte present at a physiologically relevant concentration in a biological sample, such as a body fluid sample. Because an antibody epitope is on the order of 8 amino acids, an immunoassay configured to detect a marker of interest will also detect polypeptides related to the marker sequence, so long as those polypeptides contain the epitope(s) necessary to bind to the antibody or antibodies used in the assay.

The term “related marker” as used herein with regard to a biomarker such as one of the kidney injury markers described herein refers to one or more fragments, variants, etc., of a particular marker or its biosynthetic parent that may be detected as a surrogate for the marker itself or as independent biomarkers. The term also refers to one or more polypeptides present in a biological sample that are derived from the biomarker precursor complexed to additional species, such as binding proteins, receptors, heparin, lipids, sugars, etc.

The term “positive going” marker as that term is used herein refer to a marker that is determined to be elevated in subjects suffering from a disease or condition, relative to subjects not suffering from that disease or condition. The term “negative going” marker as that term is used herein refer to a marker that is determined to be reduced in subjects suffering from a disease or condition, relative to subjects not suffering from that disease or condition.

The term “subject” as used herein refers to a human or non-human organism. Thus, the methods and compositions described herein are applicable to both human and veterinary disease. Further, while a subject is preferably a living organism, the invention described herein may be used in post-mortem analysis as well. Preferred subjects are humans, and most preferably “patients,” which as used herein refers to living humans that are receiving medical care for a disease or condition. This includes persons with no defined illness who are being investigated for signs of pathology.

Preferably, an analyte is measured in a sample. Such a sample may be obtained from a subject, or may be obtained from biological materials intended to be provided to the subject. For example, a sample may be obtained from a kidney being evaluated for possible transplantation into a subject, and an analyte measurement used to evaluate the kidney for preexisting damage. Preferred samples are body fluid samples.

The term “body fluid sample” as used herein refers to a sample of bodily fluid obtained for the purpose of diagnosis, prognosis, classification or evaluation of a subject of interest, such as a patient or transplant donor. In certain aspects, such a sample may be obtained for the purpose of determining the outcome of an ongoing condition or the effect of a treatment regimen on a condition, for example, RRT. Preferred body fluid samples include blood (including whole blood, serum, and plasma), cerebrospinal fluid, urine, saliva, sputum, pleural effusions, hemofiltrate, and ultrafiltrate. In addition, one of skill in the art would realize that certain body fluid samples would be more readily analyzed following a fractionation or purification procedure, for example, separation of whole blood into serum or plasma components.

A prognostic risk signals a probability (“a likelihood”) that a given course or outcome will occur. A level or a change in level of a prognostic indicator, which in turn is associated with an increased probability of morbidity (e.g., worsening renal function, future AKI, or death) is referred to as being “indicative of an increased likelihood” of an adverse outcome in a patient.

Marker Assays

In general, immunoassays involve contacting a sample containing or suspected of containing a biomarker of interest with at least one antibody that specifically binds to the biomarker. A signal is then generated indicative of the presence or amount (e.g., concentration) of complexes formed by the binding of polypeptides in the sample to the antibody. The signal is then related to the presence or amount of the biomarker in the sample. Numerous methods and devices are well known to the skilled artisan for the detection and analysis of biomarkers. See, e.g., U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, and The Immunoassay Handbook, David Wild, ed. Stockton Press, New York, 1994, each of which is hereby incorporated by reference in its entirety, including all tables, FIGURES and claims.

The assay devices and methods known in the art can utilize labeled molecules in various sandwich, competitive, or non-competitive assay formats, to generate a signal that is related to the presence or level/amount of the biomarker of interest. Suitable assay formats also include chromatographic, mass spectrographic, and protein “blotting” methods. Additionally, certain methods and devices, such as biosensors and optical immunoassays, may be employed to determine the presence or amount of analytes without the need for a labeled molecule. See, e.g., U.S. Pat. Nos. 5,631,171; and 5,955,377, each of which is hereby incorporated by reference in its entirety, including all tables, FIGURES and claims. One skilled in the art also recognizes that robotic instrumentation including but not limited to Beckman ACCESS®, Abbott AXSYM®, Roche ELECSYS®, Dade Behring STRATUS® systems are among the immunoassay analyzers that are capable of performing immunoassays. However, any suitable immunoassay may be utilized, for example, enzyme-linked immunoassays (ELISA), radioimmunoassays (RIAs), competitive binding assays, and the like.

Antibodies or other polypeptides may be immobilized onto a variety of solid supports for use in assays. Solid phases that may be used to immobilize specific binding members include those developed and/or used as solid phases in solid phase binding assays. Examples of suitable solid phases include membrane filters, cellulose-based papers, beads (including polymeric, latex and paramagnetic particles), glass, silicon wafers, microparticles, nanoparticles, TentaGels, AgroGels, PEGA gels, SPOCC gels, and multiple-well plates. An assay strip may be prepared by coating the antibody or a plurality of antibodies in an array on solid support. This strip may then be dipped into the test sample and then processed quickly through washes and detection steps to generate a measurable signal, such as a colored spot. Antibodies or other polypeptides may be bound to specific zones of assay devices either by conjugating directly to an assay device surface, or by indirect binding. In an example of the latter case, antibodies or other polypeptides may be immobilized on particles or other solid supports, and that solid support immobilized to the device surface.

Biological assays require methods for detection, and one of the most common methods for quantitation of results is to conjugate a detectable label to a protein or nucleic acid that has affinity for one of the components in the biological system being studied. Detectable labels may include molecules that are themselves detectable (e.g., fluorescent moieties, electrochemical labels, metal chelates, etc.) as well as molecules that may be indirectly detected by production of a detectable reaction product (e.g., enzymes such as horseradish peroxidase, alkaline phosphatase, etc.) or by a specific binding molecule which itself may be detectable (e.g., biotin, digoxigenin, maltose, oligohistidine, 2,4-dintrobenzene, phenylarsenate, ssDNA, dsDNA, etc.).

Preparation of solid phases and detectable label conjugates often comprise the use of chemical cross-linkers. Cross-linking reagents contain at least two reactive groups, and are divided generally into homofunctional cross-linkers (containing identical reactive groups) and heterofunctional cross-linkers (containing non-identical reactive groups). Homobifunctional cross-linkers that couple through amines, sulfhydryls or react non-specifically are available from many commercial sources. Maleimides, alkyl and aryl halides, alpha-haloacyls and pyridyl disulfides are thiol reactive groups. Maleimides, alkyl and aryl halides, and alpha-haloacyls react with sulfhydryls to form thiol ether bonds, while pyridyl disulfides react with sulfhydryls to produce mixed disulfides. The pyridyl disulfide product is cleavable. Imidoesters are also very useful for protein-protein cross-links. A variety of heterobifunctional cross-linkers, each combining different attributes for successful conjugation, are commercially available.

The skilled artisan will understand that the signals obtained from an immunoassay are a direct result of complexes formed between one or more antibodies and the target biomolecule (i.e., the analyte) and polypeptides containing the necessary epitope(s) to which the antibodies bind. While such assays may detect the full length biomarker and the assay result be expressed as a concentration of a biomarker of interest, the signal from the assay is actually a result of all such “immunoreactive” polypeptides present in the sample. Expression of biomarkers may also be determined by means other than immunoassays, including protein measurements (such as dot blots, western blots, chromatographic methods, mass spectrometry, etc.) and nucleic acid measurements (mRNA quantitation). This list is not meant to be limiting.

In certain aspects, the present invention provides kits for the analysis of the described biomarkers. The kit comprises reagents for the analysis of at least one test sample which comprise at least one antibody that specifically binds to one of the biomarkers disclosed herein. The kit can also include devices and instructions for performing one or more of the prognostic correlations described herein. Preferred kits will comprise an antibody pair for performing a sandwich assay, or a labeled species for performing a competitive assay, for the analyte. Preferably, an antibody pair comprises a first antibody conjugated to a solid phase and a second antibody conjugated to a detectable label, wherein each of the first and second antibodies bind a kidney injury marker. Most preferably, each of the antibodies are monoclonal antibodies. The instructions for use of the kit and performing the correlations may be in the form of labeling, which refers to any written or recorded material that is attached to, or otherwise accompanies a kit at any time during its manufacture, transport, sale or use. For example, the term labeling encompasses advertising leaflets and brochures, packaging materials, instructions, audio or video cassettes, computer discs, flash memory drives, as well as writing imprinted directly on kits.

Antibodies

The term “antibody” as used herein refers to a peptide or polypeptide derived from, modeled after or substantially encoded by an immunoglobulin gene or immunoglobulin genes, or fragments thereof, capable of specifically binding an antigen or epitope. See, e.g. Fundamental Immunology, 3rd Edition, W. E. Paul, ed., Raven Press, N.Y. (1993); Wilson (1994; J. Immunol. Methods 175:267-273; Yarmush (1992) J. Biochem. Biophys. Methods 25:85-97. The term antibody includes antigen-binding portions, i.e., “antigen binding sites,” (e.g., fragments, subsequences, complementarity determining regions (CDRs)) that retain capacity to bind antigen, including (i) a Fab fragment, a monovalent fragment consisting of the VL, VH, CL and CHI domains; (ii) a F(ab′)2 fragment, a bivalent fragment comprising two Fab fragments linked by a disulfide bridge at the hinge region; (iii) a Fd fragment consisting of the VH and CHI domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Single chain antibodies are also included by reference in the term “antibody.”

Antibodies used in the immunoassays described herein preferably specifically bind to a biomarker of the present invention. The term “specifically binds” is not intended to indicate that an antibody binds exclusively to its intended target since, as noted above, an antibody binds to any polypeptide displaying the epitope(s) to which the antibody binds. Rather, an antibody “specifically binds” if its affinity for its intended target is about greater when compared to its affinity for a non-target molecule which does not display the appropriate epitope(s). Preferably the affinity of the antibody will be at least about 5 fold, preferably 10 fold, more preferably 25-fold, even more preferably 50-fold, and most preferably 100-fold or more, greater for a target molecule than its affinity for a non-target molecule. In preferred aspects, preferred antibodies bind with affinities of at least about 10⁷ M⁻¹, and preferably between about 10⁸ M⁻¹ to about 10⁹ M⁻¹, about 10⁹ M⁻¹ to about 10¹⁰ M⁻¹, or about 10¹⁰ M⁻¹ to about 10¹² M⁻¹.

Affinity is calculated as K_{d}=k_{off}/kₒₙ (k_{off} is the dissociation rate constant, Kₒₙ is the association rate constant and K_{d} is the equilibrium constant). Affinity can be determined at equilibrium by measuring the fraction bound (r) of labeled ligand at various concentrations (c). The data are graphed using the Scatchard equation: r/c=K(n−r): where r=moles of bound ligand/mole of receptor at equilibrium; c=free ligand concentration at equilibrium; K=equilibrium association constant; and n=number of ligand binding sites per receptor molecule. By graphical analysis, r/c is plotted on the Y-axis versus r on the X-axis, thus producing a Scatchard plot. Antibody affinity measurement by Scatchard analysis is well known in the art. See, e.g., van Erp et al., J. Immunoassay 12: 425-43, 1991; Nelson and Griswold, Comput. Methods Programs Biomed. 27: 65-8, 1988.

The term “epitope” refers to an antigenic determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents.

Assay Correlations

The term “correlating” as used herein in reference to the use of biomarkers may refer to comparing the presence or level/amount (e.g., concentration) of the biomarker(s) in a subject to its presence or level/amount in persons known to benefit from treatment with continued RRT; or in persons known to improve from treatment without continued RRT. Often, this takes the form of comparing an assay result in the form of a biomarker level (e.g., a concentration) to a predetermined threshold selected to be indicative of the likelihood of some future outcome, for example, improvement of kidney function, with or without administration of continued RRT.

Selecting a threshold involves, among other things, distribution of true and false predictions/correlations at different test thresholds, and estimates of the consequences of treatment (or a failure to treat). For example, when considering administering a specific therapy which is highly efficacious and has a low level of risk, few tests are needed because clinicians can accept substantial diagnostic uncertainty. On the other hand, in situations where treatment options are less effective and riskier, clinicians often need a higher degree of diagnostic certainty. Thus, cost/benefit analysis is involved in selecting a threshold.

Suitable thresholds may be determined in a variety of ways. For example, one recommended threshold for the diagnosis of acute myocardial infarction using cardiac troponin is the 97.5^{th} percentile of the concentration seen in a normal population. In some instances, the threshold may be selected from a population of subjects likely not to benefit from treatment with continued RRT such that, for example, the threshold excludes a certain proportion (e.g., a majority) of those subjects (e.g., the threshold is set at the 70^{th}, 80^{th}, 85^{th}, 90^{th}, 95^{th}, 97.5^{th}, 98^{th}, 99^{th}, 99.5^{th} or 99.9^{th} percentile) from being classified as having an increased likelihood of benefitting from treatment with continued RRT. In some instances, the threshold may be selected from a population of subjects likely to benefit from treatment with continued RRT such that, for example, the threshold includes a certain proportion (e.g., a majority) of those subjects (e.g., the threshold is set at the 0.1^{th}, 0.5^{th}, 1^{st}, 2^{nd}, 2.5^{th}, 5^{th}, 10^{th}, 15^{th}, 20^{th}, 25^{th}, or 30^{th} percentile) in being classified as having an increased likelihood of benefitting from treatment with continued RRT.

Another method may be to look at serial samples (e.g., 2, 3, 4, 5, or more temporally spaced samples) from the same patient, where a prior “baseline” result is used to monitor for temporal changes in a biomarker level. For example, a baseline level of a biomarker may be established from one or more (e.g., an average) measurements from a subject before the subject is placed on RRT and/or from one or more (e.g., an average) measurements from a subject after the subject has been placed on RRT. In some instances, the baseline level may be established from one or more measurements over a specific time frame (e.g., within 1, 2, 3, 4, or 5 days before initiating RRT and/or within 1, 2, 3, 4, or 5 days after initiating RRT). Increases in a positive-going biomarker level measured over the baseline level during RRT may be indicative of an increased likelihood of benefitting from treatment with continued RRT and/or decreases in a positive-going biomarker level measured over the baseline level during RRT may be indicative of a decreased likelihood of benefiting from treatment with continued RRT. In some instances, RRT may be continued as long as one or more biomarker levels are remaining steady, continuing to increase, or continuing to increase at a certain rate relative to a baseline level. In some instances, RRT may be discontinued if one or more biomarker levels are remaining steady, continuing to decrease, or continuing to decrease at a certain rate relative to a baseline level. Alternatively, the opposite relationship may be applicable for a negative going marker. The trend relative to baseline may be determined or evaluated from one or more measurements during RRT or for all measurements over a duration of time of receiving RRT (e.g., over 1, 2, or 3 days).

Population studies may also be used to select a decision threshold. Receiver Operating Characteristic (“ROC”) arose from the field of signal detection theory developed during World War II for the analysis of radar images, and ROC analysis is often used to select a threshold able to best distinguish a “diseased” subpopulation from a “nondiseased” subpopulation. A false positive in this case occurs when the person tests positive, but actually does not have the disease. A false negative, on the other hand, occurs when the person tests negative, suggesting they are healthy, when they actually do have the disease. To draw a ROC curve, the true positive rate (TPR) and false positive rate (FPR) are determined as the decision threshold is varied continuously. Since TPR is equivalent with sensitivity and FPR is equal to 1—specificity, the ROC graph is sometimes called the sensitivity vs (1—specificity) plot. A perfect test will have an area under the ROC curve of 1.0; a random test will have an area of 0.5. A threshold is selected to provide an acceptable level of specificity and sensitivity.

In this context, “diseased” is meant to refer to a population having one characteristic (the presence of a disease or condition or the occurrence of some outcome) and “nondiseased” is meant to refer to a population lacking the characteristic. While a single decision threshold is the simplest application of such a method, multiple decision thresholds may be used. For example, below a first threshold, the absence of disease may be assigned with relatively high confidence, and above a second threshold the presence of disease may also be assigned with relatively high confidence. Between the two thresholds may be considered indeterminate. This is meant to be exemplary in nature only.

According to certain aspects of the disclosure, the threshold(s) may be used to decide whether the subject is likely to benefit from RRT. In an example of a single decision threshold, above the threshold for a positive going marker may indicate that the subject is assigned to a group which will benefit from RRT while at or below the threshold may indicate that the subject is assigned to a group which will not benefit from RRT.

Alternatively, the opposite relationship may be applicable for a negative going marker.

Specifically, at or below the threshold may indicate that the subject is assigned to a group which will benefit from RRT while above the threshold may indicate that the subject is assigned to a group which will not benefit from RRT. Furthermore, like the example of diseased compared to nondiseased, a multiple decision threshold may be used to assess whether a subject is likely to benefit from RRT. For example, below a first threshold, the likelihood that the subject will not benefit from RRT may be assigned with relatively high confidence, and above a second threshold the likelihood that the subject will benefit from RRT may also be assigned with relatively high confidence. Between the two thresholds may be considered indeterminate. This is meant to be exemplary in nature only.

According to various aspects, one or more of the biomarkers disclosed herein (e.g., CCL14 and/or KLK14) are used, individually or in panels comprising a plurality of biomarkers, for correlating assay results to a subject classification (e.g. likelihood of an outcome in response to RRT). Classifications may be made by threshold comparisons as described elsewhere herein. In addition to threshold comparisons, other methods for correlating assay results are known in the art. These methods may use two or more variables together in combination (e.g., two or more of the biomarkers disclosed herein and/or one or more of the biomarkers with one or more other clinical indicia disclosed elsewhere herein) to correlate the assay result(s). These methods include, for example, decision tree analysis, random forests analysis, n-of-m (number positive) analysis, neural networks, Bayesian model, and rule sets. According to some specific aspects, one or more of these biomarkers and/or clinical indicia are used together in combination by combining them into a single composite value. Methods for combining one or more biomarkers and/or clinical indicia into a single composite value include, for example, multiplication/division (e.g., a product or ratio of biomarkers), addition/subtraction, logistic regression, loglinear modeling, and use of linear discriminants. In these methods, a composite result may be treated as if it is itself a marker; that is, a threshold may be determined for the composite result as described herein for individual markers, and the composite result for an individual subject compared to this threshold. Some methods of correlating assay results, such as, for example, logistic regression, decision trees, rule sets, Bayesian methods, and neural network methods can produce probability values representing the degree to which a subject belongs to one classification out of a plurality of classifications.

Measures of test accuracy may be obtained as described in Fischer et al., Intensive Care Med. 29: 1043-51, 2003, and used to determine the effectiveness of a given biomarker. These measures include sensitivity and specificity, predictive values, likelihood ratios, diagnostic odds ratios, and ROC curve areas. The area under the curve (“AUC”) of a ROC plot is equal to the probability that a classifier will rank a randomly chosen positive instance higher than a randomly chosen negative one. The area under the ROC curve may be thought of as equivalent to the Mann-Whitney U test, which tests for the median difference between scores obtained in the two groups considered if the groups are of continuous data, or to the Wilcoxon test of ranks.

As discussed above, suitable tests may exhibit one or more of the following results on these various measures: a specificity of greater than 0.5, preferably at least 0.6, more preferably at least 0.7, still more preferably at least 0.8, even more preferably at least 0.9 and most preferably at least 0.95, with a corresponding sensitivity greater than 0.2, preferably greater than 0.3, more preferably greater than 0.4, still more preferably at least even more preferably 0.6, yet more preferably greater than 0.7, still more preferably greater than 0.8, more preferably greater than 0.9, and most preferably greater than 0.95; a sensitivity of greater than 0.5, preferably at least 0.6, more preferably at least 0.7, still more preferably at least 0.8, even more preferably at least 0.9 and most preferably at least 0.95, with a corresponding specificity greater than 0.2, preferably greater than 0.3, more preferably greater than 0.4, still more preferably at least 0.5, even more preferably yet more preferably greater than 0.7, still more preferably greater than 0.8, more preferably greater than 0.9, and most preferably greater than 0.95; at least 75% sensitivity, combined with at least 75% specificity; a ROC curve area (AUC) of greater than 0.5, preferably at least 0.6, more preferably 0.7, still more preferably at least 0.75, even more preferably at least 0.8, still even more preferably at least 0.9, and most preferably at least 0.95; an odds ratio different from 1, preferably at least about 2 or more or about 0.5 or less, more preferably at least about 3 or more or about 0.33 or less, still more preferably at least about 4 or more or about 0.25 or less, even more preferably at least about 5 or more or about 0.2 or less, and most preferably at least about 10 or more or about 0.1 or less; a positive likelihood ratio (calculated as sensitivity/(1-specificity)) of greater than 1, at least 2, more preferably at least 3, still more preferably at least 5, and most preferably at least 10; and or a negative likelihood ratio (calculated as (1-sensitivity)/specificity) of less than 1, less than or equal to 0.5, more preferably less than or equal to 0.3, and most preferably less than or equal to 0.1

C-C motif chemokine 14 (CCL14) may function as a biomarker correlated to likelihood that a subject suffering from AKI or persistent AKI will improve in response to treatment with continued RRT. CCL14 is a member of the chemokine family of small molecules that were initially recognized for roles in leukocyte chemotaxis and are implicated in tissue injury and repair processes. CCL14 binds with high affinity to the chemokine receptors CCR1 and CCRS and lower affinity to CCR3. (Detheux M, Standker L, Vakili J, Munch J, Forssmann U, Adermann K, et al. J Exp Med. 2000; 192(10):1501-8.) CCL14 is an important chemokine for monocyte/macrophage recruitment and is associated with pro-inflammatory chemotaxis in a variety of diseases including rheumatoid arthritis, multiple sclerosis, and lupus (Rump L, Mattey D L, Kehoe O, Middleton J. Cytokine. 2017; 97:133-40; Vyshkina T, Sylvester A, Sadiq S, Bonilla E, Perl A, Kalman B. J Neuroimmunol. 2008;200(1-2):145-52).

Kallikrein 14 (KLK14) may function as a biomarker correlated to likelihood that a subject suffering from AKI or persistent AKI will improve in response to treatment with continued RRT. Kallikrein 14 is a member of a subfamily of serine proteases which has a variety of physiological functions. (Borgono C A, Michael I P, and Diamandis EEP. Mol Cancer Res. 2004; 2(5):257-80). It may perform these functions by activating or inactivating proteinase-activated receptors. (Oikonomopoulou K, Hansen K K, Saifeddine M, Tea I, Blaber M, Blaber S I, Scarisbrick I, Andrade-Gordon P, Cottrell G S, Bunnett Diamandis E P, and Hollenberg M D. J. Biol. Chem. (2006)281:32095-112). Kallikrein 14 is thought to participate in disease processes, including breast cancer. (Fritzsche F, Gansukh T, Borgono C A, Burkhardt M, Pahl S, Mayordomo E, Winzer K-J, Weichert W, Denkert C, Jung K, Stephan C, Dietel, M, Diamandis E P, Dahl E, and Kristiansen G. Br. J. Cancer 2006; 94(4):540-7).

Other clinical indicia which may be combined with the biomarker assay result(s) of the present invention includes demographic information (e.g., weight, sex, age, race), medical history (e.g., family history, type of surgery, pre-existing disease such as aneurism, congestive heart failure, preeclampsia, eclampsia, diabetes mellitus, hypertension, coronary artery disease, proteinuria, renal insufficiency, or sepsis, type of toxin exposure such as NSAIDs, cyclosporines, tacrolimus, aminoglycosides, foscarnet, ethylene glycol, hemoglobin, myoglobin, ifosfamide, heavy metals, methotrexate, radiopaque contrast agents, or streptozotocin), clinical variables (e.g., blood pressure, temperature, respiration rate), risk scores (APACHE score, PREDICT score, TIMI Risk Score for UA/NSTEMI, Framingham Risk Score), a urine total protein measurement, a glomerular filtration rate, an estimated glomerular filtration rate, a urine production rate, a serum or plasma creatinine concentration, a renal papillary antigen 1 (RPA1) measurement; a renal papillary antigen 2 (RPA2) measurement; a urine creatinine concentration, a fractional excretion of sodium, a urine sodium concentration, a urine creatinine to serum or plasma creatinine ratio, a urine specific gravity, a urine osmolality, a urine urea nitrogen to plasma urea nitrogen ratio, a plasma BUN to creatinine ratio, and/or a renal failure index calculated as urine sodium/(urine creatinine/plasma creatinine). Other clinical indicia which may be combined with the biomarker assay result(s) of the present invention include clinical indicia which may be specific to RRT, such as measures of serum potassium, serum bicarbonate, serum sodium, serum phosphate, blood pH, arterial blood gas (PaO₂, PaCO₂), fluid input and output (fluid balance), and urine output, as well as clinical statuses related to pulmonary edema, encephalopathy, pericarditis, and receipt of vasoactive agents. Other measures of renal function which may be combined with the kidney injury marker assay result(s) are described hereinafter and in Harrison's Principles of Internal Medicine, 17^{th} Ed., McGraw Hill, New York, pages 1741-1830, and Current Medical Diagnosis & Treatment 2008, 47^{th} Ed, McGraw Hill, New York, pages 785-815, each of which are hereby incorporated by reference in their entirety.

Treatment

Renal replacement therapy (RRT) is an option for management of patients suffering from renal dysfunction, including AKI, AKD, or CKD. RRT includes renal transplant as well as various types of dialysis. Dialysis filters and removes waste products, electrolytes, and water from the body similar to the function of the kidney. Multiple dialysis protocols are in use. The different types of dialysis generally fall within the categories of hemodialysis and peritoneal dialysis. Hemodialysis clears solutes from the blood by diffusion across an artificial membrane using a concentration gradient. Peritoneal dialysis, which uses the peritoneum as a semi-permeable membrane to remove solvents, is also in clinical use. Unlike hemodialysis which directly filters the blood, peritoneal dialysis includes injecting fluid into the peritoneal cavity. The peritoneum acts as a filter and fluid is then removed with accompanying waste products, electrolytes, and excess water. Timing of dialysis has been shown to be relevant to the patient outcome. Reviewed by Pannu N and Noel Gibney R T. Ther Clin Risk Manag. 2005; 1(2):141-50, which is hereby incorporated by reference in its entirety. More specific dialysis procedures include intermittent renal replacement therapies (IRRTs) and continuous renal replacement therapies (CRRTs). IRRTs include intermittent hemodialysis, intermittent hemofiltration, and intermittent hemodiafilitration. CRRTs include continuous hemofiltration and continuous hemodiafiltration. There are also hybrid dialysis protocols called prolonged intermittent renal replacement therapies (PIRRTs). These include sustained low-efficiency dialysis (SLED) and extended-duration dialysis (EDD). Some types may be performed at the subject's home or during travel while some require a clinical setting with the assistance of healthcare professionals. The methods described herein may be used to determine that the type of RRT should be altered. For example, in some implementations, if it is determined that the subject may not benefit from treatment with continued RRT, a practitioner may decide to switch the subject to a less aggressive form of RRT rather than discontinue RRT altogether.

According to the disclosed methods, subjects being assessed for treatment with continued RRT or discontinuation of RRT may be assigned a relatively increased likelihood or a relatively decreased likelihood of benefiting from treatment with continued RRT based on one or more assay results for one or more biomarkers described elsewhere herein (e.g., treatment with RRT lasting more than 1, 2, 3, or more days from measurement of the one or more biomarkers). Subjects may accordingly be treated with continued or discontinued RRT. More specifically, depending on whether the subject's biomarker level(s) (e.g., CCL14, KLK14, and/or others disclosed herein) is above a threshold biomarker level or at or below a threshold biomarker level, the subject may be treated with continued or discontinued RRT, respectively. The continuation of RRT may occur with the expectation that the extended RRT will benefit the subject, including, but not limited to, improved renal function (e.g., decreased serum creatinine levels, increased urine output, and/or recovering to a less severe stage of AKI). Treatment with discontinued RRT may be undertaken with the expectation that the subject will not benefit from treatment with continued RRT. A subject who will not benefit from treatment with continued RRT may be one whose health status will improve without continued RRT. This improvement may comprise improved renal function, among other health status improvements. Alternatively, a subject who will not benefit from treatment with continued RRT may be a subject who will not experience an improvement in health status, with or without treatment with continued RRT. In the latter case, the risks and use of resources associated with treatment with continued RRT likely outweigh the benefit to the subject. In some embodiments, a subject who has successfully been treated with discontinued RRT may be considered a subject who would not have benefited from treatment with continued RRT. Discontinuation of RRT may be considered successful if the subject goes without receiving RRT, for example, for more than 1, 2, 3, 4, or 5 days without needing to be placed back on RRT and if the subject does not suffer death and/or any other major adverse kidney events during such a time period.

Discontinuing RRT may comprise one or more steps such as disconnecting or removing one or more lines, needles, ports, dialyzers, dialysis bags, waste bags, and/or pumps from each other and/or from a subject. Discontinuing RRT may comprise providing orders to a health care practitioner or to a subject on dialysis to discontinue RRT. Discontinuing RRT may comprise entering orders into an electronic patient management system to discontinue RRT or to revise a patient dialysis schedule to remove or cancel scheduled dialysis treatments. In some embodiments, alternative (e.g., more conservative/less aggressive) treatments may be administered to a subject discontinuing dialysis. For example, the subject may be treated by modifying administration of compounds known to be damaging to the kidney by adjusting the amount or selection of the compound (e.g., withdrawing or reducing delivery of compounds that are known to be damaging to the kidney), delaying or avoiding procedures that are known to be damaging to the kidney, modifying diuretic administration, initiating goal directed therapy, etc. The skilled artisan is aware of appropriate treatments for numerous diseases discussed in relation to the methods of diagnosis described herein. See, e.g., Merck Manual of Diagnosis and Therapy, 17^{th} Ed. Merck Research Laboratories, Whitehouse Station, N J, 1999, hereby incorporated by reference in its entirety. Diuretics are used in the setting of acute kidney injury (AKI) to optimize fluid management and aid in the management of electrolyte disorders. However, diuretic therapy can also have adverse effects including volume depletion, hypotension, decreased cardiac output, and worsening renal function. Decisions associated with administration, dosing, or withdrawal of diuretics depend not only on renal status but other aspects of patient status, such as fluid balance. Such factors which determine the appropriate use of diuretics in patients with or at risk for developing AKI are understood by those skilled in the art, as described, for example in Cerda, J. “Loop Diuretics in Acute Kidney Injury” Encyclopedia of Intensive Care Medicine, 2012 Ed, p 1337-1341, herein incorporated by reference in its entirety.

Administering continued RRT may comprise one or more steps such as connecting or installing one or more lines, needles, ports, dialyzers, dialysis bags, waste bags, and/or pumps to each other and/or to a subject. Administering continued RRT may comprise operating and/or monitoring a dialysis system. Administering continued RRT may comprise monitoring a subject (e.g., a subject's vitals) during dialysis. Administering continued RRT may comprise providing orders to a health care practitioner or to a subject on dialysis to proceed with RRT. Administering continued RRT may comprise entering orders into an electronic patient management system to continue RRT or to revise a patient dialysis schedule to schedule or add dialysis treatments. A subject who is determined may benefit from treatment with continued RRT may be retested according to methods described herein (e.g., using the same one or more biomarkers and/or different biomarkers) in approximately 12, 24, 36, 48, 60, 72, 84, 96, or more hours from the initial or previous test to determine if RRT should be further continued. Bed rest or a diet or medication appropriate for subjects on continued dialysis may be prescribed for a subject receiving continued dialysis. The alternative treatment options described above for discontinuing RRT may be administered to a subject treated with continued RRT in addition to the continued RRT.

One skilled in the art readily appreciates that the present invention is well adapted to carry out the objects and obtain the ends and advantages mentioned, as well as those inherent therein. The examples provided herein are representative of preferred embodiments, are exemplary, and are not intended as limitations on the scope of the invention.

Body fluid samples referenced in the following Examples were collected as described in in Hoste E, Bihorac A, Al-Khafaji A, Ortega L M, Ostermann M, Haase M, Zacharowski K, et al. “Identification and Validation of Biomarkers of Persistent Acute Kidney Injury: The Ruby Study.” Intensive Care Med. 2020; 46:943-53, which is hereby incorporated by reference in its entirety. While subjects who were currently receiving RRT were excluded from the Ruby Study, the subjects in the Examples below were those who later began RRT and for which samples were first collected after RRT was initiated.

### EXAMPLES

### Example 1. Acutely Ill Subject Sample Collection

The objective of this study was to collect blood and urine samples from patients with known moderate or severe AKI (KDIGO AKI stage 2 or 3) at the time of enrollment. The study enrolled approximately 300 critically ill, hospitalized adult subjects within 36 hours of reaching KDIGO AKI stage 2 criteria.

To be enrolled in the study, each subject must have met all the following inclusion criteria and none of the following exclusion criteria:

### Inclusion Criteria

Males and females 21 years of age or older; receiving care in a hospital or health care facility for at least 48 hours after enrollment; use of indwelling urinary catheter as standard care at the time of enrollment; subject must have acute kidney injury (KDIGO stage 2 or 3) at the time of the first sample collection; first sample must be collected within 36 hours of meeting KDIGO stage 2 criteria; written informed consent provided by patient or legally authorized representative. All subjects were hospitalized for at least one of the following list of indications: congestive heart failure, diabetes mellitus, hypertension, coronary artery disease, renal insufficiency, glomerular filtration below the normal range, cirrhosis, serum creatinine above the normal range, sepsis, injury to renal function, reduced renal function, acute kidney injury (KDIGO Stage 1, 2, or 3), respiratory disease, surgery, cardiovascular disease, and neurological disease. Comorbidities include chronic kidney disease, diabetes mellitus, congestive heart failure, coronary artery disease, hypertension, chronic obstructive pulmonary disease, and cancer. Where data on surgery or trauma was collected, subjects had undergone surgery or trauma within 3 days of enrollment. The first samples were collected within 12 hours of enrollment. Consequently, subjects who had experienced surgery or trauma had done so within 3.5 days (84 hours) prior to the first sample collections.

Exclusion Criteria Prior kidney transplantation; comfort-measures-only status; already receiving dialysis (either acute or chronic) or in imminent need of dialysis at the time of enrollment; history of human immunodeficiency virus (HIV) or hepatitis virus (based upon available medical records) infections; special populations, such as pregnant women, prisoners, or institutionalized individuals; patient meets any of the following: (i) active bleeding with an anticipated need for >4 units PRBC in a day, (ii) hemoglobin <7 g/dL, (iii) any other condition in the physician's opinion would contraindicate drawing serial blood samples for clinical study purposes.

Once informed consent was obtained and eligibility confirmed, each patient had blood samples collected for processing to plasma and serum and a urine specimen collected twice daily for the first 3 days and then daily through day 7 while the patient was in the hospital, as follows: (i) an EDTA-anticoagulated venous or arterial blood sample (10m L) was drawn for processing to plasma; (ii) a venous or arterial blood sample without anticoagulant (3 mL) was drawn for processing to serum; (iii) a urine sample (50m L) was obtained and processed.

### Example 2. Use of an Analyte to Evaluate Patients Who are on Renal Replacement Therapy (RRT) for the Discontinuation of Treatment

Patients from Example 1 who began receiving renal replacement therapy (RRT) within 7 days of enrolling in the study of Example 1 were included in the following analysis. Blood samples and urine samples were collected from each patient at enrollment in the study of Example 1, and at every 12 hours up to day 3, and then every 24 hours thereafter up to day 7 while the subject was hospitalized. Analyte concentrations in collected urine and/or plasma samples were measured for the days during which the patient was receiving RRT by standard immunoassay methods using commercially available assay reagents or in a lateral flow assay format.

Renal replacement therapy status was determined from the patient's medical record. The status was recorded daily from enrollment in the study of Example 1 to 6 days after and as the treatment was performed from 7 to 90 days after. Two cohorts were defined to represent a “discontinuation” and a “not discontinuation” population. “Discontinuation” indicates those patients whose RRT treatment ended within 1, 2 or 3 days after the time of sample collection, and who survived and did not receive additional RRT treatment for at least 2 days after RRT treatment ended. This time frame for successful discontinuation of RRT has been documented by Yoshida T, Matsuura, R, Komaru Y. et al. Nephrol. 2019: 24(3); 287-93, which is hereby incorporated by reference in its entirety. “Not discontinuation” indicates those patients whose RRT treatment persisted beyond 1, 2 or 3 days after the time of sample collection, or who died within 2 days after RRT treatment ended, or who received additional RRT within 2 days after RRT treatment ended.

The ability to distinguish the “discontinuation” and “not discontinuation” cohorts was determined using a receiver operating characteristic (ROC) analysis with the analyte concentrations from the different sample collections. The performance of the analyte was assessed by the area under the ROC curve (AUC). All markers disclosed herein were positive going markers (AUC >0.5 correlates to continuation (i.e. “not discontinuation”) of RRT). The AUCs and number of discontinuation, not discontinuation, and total samples for each marker are reported in Tables 1a, 2a, and 3a below for discontinuation of RRT within 1, 2, or 3 days of sample collection, respectively.

The ability of an analyte to distinguish the “discontinuation” and “not discontinuation” cohorts was further characterized by the sensitivity (true positive rate), specificity (true negative rate) and diagnostic odds ratio (ratio of the number of true positive to false negative relative to the ratio of the number of false positive to true negative). A true positive referred to the analyte testing positive for patients in the “not discontinuation” population, while a true negative referred to the analyte testing negative for patients in the “not discontinuation” population. Analyte concentrations at the 25^{th} 50^{th} (median), and 75^{th} percentiles of the observed range of concentrations were used as decision thresholds where values above the threshold were considered a positive test result while those at or below the threshold were considered a negative test result. The threshold, sensitivity, specificity, and odds ratio defined by the 25^{th} percentile for each marker are reported in Tables 1 b, 2b, and 3b for discontinuation of RRT within 1, 2, or 3 days of sample collection, respectively; the threshold, sensitivity, specificity, and odds ratio defined by the 50^{th} percentile (median) for each marker are reported in Tables 1c, 2c, and 3c for discontinuation of RRT within 1, 2, or 3 days of sample collection, respectively; the threshold, sensitivity, specificity, and odds ratio defined by the 75^{th} percentile for each marker are reported in Tables 1d, 2d, and 3d for discontinuation of RRT within 1, 2, or 3 days of sample collection, respectively. Confidence intervals were calculated by the bootstrap method. “Inf” represents positive infinity. In cases where the point estimate of the specificity was 100%, a lower bound of the odds ratio (reported as “>lower bound”) was computed by adding 1 to the number of false positive cases.

TABLE 1a
AUC and the number of “discontinuation”, “not discontinuation” and total
samples where RRT treatment discontinuation, if any, starts within 1 day of sample collection.

| | Swiss-Prot | Urine or | | # | # Not | Total # |
|---|---|---|---|---|---|---|
| Preferred Name | Entry Number | Plasma | AUC (95% CI) | Discontinuation | Discontinuation | samples |
| Antileukoproteinase | P03973 | U | 0.78 (0.64-0.93) | 7 | 33 | 40 |
| Cathepsin B | P07858 | U | 0.71 (0.56-0.84) | 13 | 64 | 77 |
| C-C motif chemokine 1 | P22362 | U | 0.73 (0.58-0.86) | 13 | 66 | 79 |
| C-C motif chemokine 14 | Q16627 | U | 0.76 (0.63-0.87) | 13 | 65 | 78 |
| C-C motif chemokine 16 | O15467 | U | 0.73 (0.58-0.86) | 13 | 63 | 76 |
| C-C motif chemokine 23 | P55773 | U | 0.73 (0.60-0.85) | 13 | 66 | 79 |
| C-C motif chemokine 24 | O00175 | U | 0.71 (0.55-0.86) | 13 | 66 | 79 |
| C-C motif chemokine 28 | Q9NRJ3 | U | 0.77 (0.56-0.98) | 7 | 33 | 40 |
| Chitinase-3-like protein 1 | P36222 | U | 0.76 (0.60-0.90) | 13 | 66 | 79 |
| C-X-C motif chemokine 2 | P19875 | U | 0.76 (0.61-0.90) | 13 | 66 | 79 |
| C-X-C motif chemokine 9 | Q07325 | U | 0.72 (0.58-0.85) | 13 | 66 | 79 |
| Dickkopf-related protein 1 | O94907 | U | 0.76 (0.62-0.88) | 13 | 66 | 79 |
| Elafin | P19957 | U | 0.78 (0.60-0.94) | 7 | 33 | 40 |
| Fatty acid-binding protein, adipocyte | P15090 | P | 0.75 (0.58-0.90) | 14 | 78 | 92 |
| Follistatin-related protein 3 | O95633 | P | 0.74 (0.59-0.87) | 14 | 78 | 92 |
| Hepatocyte growth factor-like protein | P26927 | U | 0.77 (0.63-0.89) | 13 | 66 | 79 |
| Insulin-like growth | P18065 | U | 0.83 (0.70-0.94) | 12 | 63 | 75 |
| factor-binding protein 2 | | | | | | |
| Insulin-like growth | P22692 | U | 0.75 (0.60-0.88) | 13 | 64 | 77 |
| factor-binding protein 4 | | | | | | |
| Insulin-like growth | Q16270 | U | 0.57 (0.40-0.76) | 12 | 60 | 72 |
| factor-binding protein 7 | | | | | | |
| Insulin-like growth | Q16270 | P | 0.61 (0.47-0.76) | 14 | 78 | 92 |
| factor-binding protein 7 | | | | | | |
| Kallikrein-14 | Q9P0G3 | U | 0.88 (0.77-0.97) | 6 | 31 | 37 |
| Metalloproteinase inhibitor 1 | P01033 | U | 0.80 (0.68-0.91) | 13 | 66 | 79 |
| Metalloproteinase inhibitor 2 | P16035 | U | 0.78 (0.62-0.92) | 13 | 66 | 79 |
| Metalloproteinase inhibitor 4 | Q99727 | U | 0.79 (0.66-0.90) | 13 | 66 | 79 |
| Neutrophil gelatinase- | P80188 | U | 0.79 (0.65-0.91) | 13 | 66 | 79 |
| associated lipocalin | | | | | | |
| Neutrophil gelatinase- | P80188 | P | 0.70 (0.55-0.84) | 14 | 78 | 92 |
| associated lipocalin | | | | | | |
| Nidogen-1 | P14543 | U | 0.72 (0.57-0.86) | 13 | 66 | 79 |
| OX-2 membrane glycoprotein | P41217 | U | 0.76 (0.64-0.86) | 13 | 66 | 79 |
| Pro-interleukin-16 | Q14005 | U | 0.78 (0.65-0.89) | 13 | 66 | 79 |
| Prolactin | P01236 | P | 0.77 (0.64-0.88) | 14 | 78 | 92 |
| Renin | P00797 | U | 0.79 (0.68-0.89) | 13 | 65 | 78 |
| Tissue factor pathway inhibitor | P10646 | U | 0.72 (0.59-0.86) | 7 | 33 | 40 |
| Tumor necrosis factor | O14763 | P | 0.77 (0.56-0.95) | 8 | 46 | 54 |
| receptor superfamily member 10B | | | | | | |
| Tumor necrosis factor | Q9Y5U5 | U | 0.79 (0.66-0.89) | 13 | 64 | 77 |
| receptor superfamily member 18 | | | | | | |
| Tumor necrosis factor | O95407 | P | 0.76 (0.63-0.88) | 14 | 77 | 91 |
| receptor superfamily member 6B | | | | | | |
| WNT1-inducible-signaling | O95388 | U | 0.73 (0.55-0.88) | 13 | 65 | 78 |
| pathway protein 1 | | | | | | |

TABLE 1b
Sensitivity, specificity and odds ratio determined by a threshold at the 25th percentile of the range of
analyte concentrations. RRT treatment discontinuation, if any, starts within 1 day of sample collection.

| | Swiss-Prot | Urine or | | | Sensitivity | Specificity | Odds Ratio |
|---|---|---|---|---|---|---|---|
| Preferred Name | Entry Number | Plasma | Unit | Threshold | (95% CI) | (95% CI) | (95% CI) |
| Antileukoproteinase | P03973 | U | ng/ml | 142 | 82% | 57% | 6 |
| | | | | | (66%-95%) | (29%-88%) | (1.7-80) |
| Cathepsin B | P07858 | U | ng/ml | 1.92 | 69% | 54% | 2.6 |
| | | | | | (54%-82%) | (27%-82%) | (0.73-11) |
| C-C motif chemokine 1 | P22362 | U | pg/ml | 0.00114 | 86% | 38% | 4 |
| | | | | | (76%-95%) | (13%-67%) | (0.89-18) |
| C-C motif chemokine 14 | Q16627 | U | ng/mL | 6.3 | 69% | 62% | 3.6 |
| | | | | | (57%-82%) | (35%-88%) | (1.1-18) |
| C-C motif chemokine 16 | O15467 | U | pg/mL | 15.5 | 79% | 54% | 4.5 |
| | | | | | (67%-91%) | (27%-79%) | (1.3-18) |
| C-C motif chemokine 23 | P55773 | U | pg/mL | 14.4 | 73% | 62% | 4.3 |
| | | | | | (59%-85%) | (38%-87%) | (1.5-18) |
| C-C motif chemokine 24 | O00175 | U | pg/ml | 21.1 | 80% | 46% | 3.5 |
| | | | | | (67%-92%) | (21%-73%) | (0.94-16) |
| C-C motif chemokine 28 | Q9NRJ3 | U | pg/mL | 34.8 | 73% | 71% | 6.7 |
| | | | | | (59%-85%) | (40%-100%) | (1.7-Inf) |
| Chitinase-3-like protein 1 | P36222 | U | ng/ml | 9.76 | 82% | 46% | 3.9 |
| | | | | | (69%-93%) | (18%-73%) | (0.99-16) |
| C-X-C motif chemokine 2 | P19875 | U | pg/mL | 19.7 | 80% | 62% | 6.5 |
| | | | | | (70%-90%) | (33%-87%) | (2-31) |
| C-X-C motif chemokine 9 | Q07325 | U | pg/ml | 385 | 73% | 62% | 4.3 |
| | | | | | (60%-85%) | (36%-87%) | (1.4-20) |
| Dickkopf-related protein 1 | O94907 | U | ng/ml | 0.26 | 80% | 46% | 3.5 |
| | | | | | (68%-91%) | (21%-73%) | (0.96-15) |
| Elafin | P19957 | U | ng/ml | 7.51 | 79% | 57% | 5 |
| | | | | | (66%-91%) | (17%-89%) | (1.1-44) |
| Fatty acid-binding protein, | P15090 | P | ng/mL | 123 | 73% | 64% | 4.9 |
| adipocyte | | | | | (60%-85%) | (38%-90%) | (1.5-26) |
| Follistatin-related protein 3 | O95633 | P | ng/mL | 55.3 | 72% | 57% | 3.4 |
| | | | | | (58%-86%) | (30%-82%) | (1-13) |
| Hepatocyte growth factor-like | P26927 | U | ng/mL | 0.204 | 80% | 69% | 9.2 |
| protein | | | | | (68%-91%) | (42%-92%) | (2.5-64) |
| Insulin-like growth factor- | P18065 | U | ng/mL | 117 | 73% | 75% | 8.1 |
| binding protein 2 | | | | | (58%-87%) | (50%-100%) | (2.5-Inf) |
| Insulin-like growth factor- | P22692 | U | ng/mL | 143 | 75% | 54% | 3.5 |
| binding protein 4 | | | | | (61%-88%) | (25%-80%) | (0.99-15) |
| Insulin-like growth factor- | Q16270 | U | ng/ml | 318 | 80% | 23% | 1.2 |
| binding protein 7 | | | | | (68%-91%) | (7%-45%) | (0.21-5.2) |
| Insulin-like growth factor- | Q16270 | P | ng/ml | 158 | 80% | 23% | 1.2 |
| binding protein 7 | | | | | (68%-91%) | (7%-45%) | (0.21-5.2) |
| Kallikrein-14 | Q9P0G3 | U | pg/mL | 10.5 | 84% | 83% | 26 |
| | | | | | (72%-95%) | (50%-100%) | (5.5-Inf) |
| Metalloproteinase inhibitor 1 | P01033 | U | ng/mL | 11.4 | 80% | 46% | 3.5 |
| | | | | | (69%-90%) | (21%-73%) | (0.9-15) |
| Metalloproteinase inhibitor 2 | P16035 | U | ng/mL | 12.4 | 89% | 54% | 9.8 |
| | | | | | (81%-97%) | (27%-81%) | (2.9-53) |
| Metalloproteinase inhibitor 4 | Q99727 | U | pg/ml | 27.4 | 80% | 54% | 4.8 |
| | | | | | (68%-91%) | (27%-82%) | (1.4-22) |
| Neutrophil gelatinase- | P80188 | U | ng/mL | 308 | 85% | 62% | 9 |
| associated lipocalin | | | | | (74%-94%) | (33%-86%) | (2.9-46) |
| Neutrophil gelatinase- | P80188 | P | ng/mL | 451 | 85% | 62% | 9 |
| associated lipocalin | | | | | (74%-94%) | (33%-86%) | (2.9-46) |
| Nidogen-1 | P14543 | U | ng/ml | 64.5 | 85% | 54% | 6.5 |
| | | | | | (74%-94%) | (27%-80%) | (1.9-32) |
| OX-2 membrane glycoprotein | P41217 | U | pg/mL | 0.00753 | 74% | 46% | 2.5 |
| | | | | | (61%-86%) | (20%-73%) | (0.65-9.8) |
| Pro-interleukin-16 | Q14005 | U | pg/ml | 0.0318 | 76% | 46% | 2.7 |
| | | | | | (64%-86%) | (20%-73%) | (0.71-11) |
| Prolactin | P01236 | P | ng/mL | 26.1 | 65% | 71% | 4.7 |
| | | | | | (51%-78%) | (47%-93%) | (1.6-27) |
| Renin | P00797 | U | ng/mL | 8.29 | 71% | 62% | 3.9 |
| | | | | | (58%-83%) | (38%-86%) | (1.5-15) |
| Tissue factor pathway | P10646 | U | pg/ml | 327 | 76% | 43% | 2.3 |
| inhibitor | | | | | (58%-91%) | (25%-75%) | (0.64-17) |
| Tumor necrosis factor | O14763 | P | pg/ml | 282 | 80% | 63% | 6.9 |
| receptor superfamily member 10B | | | | | (65%-92%) | (25%-100%) | (1.3-Inf) |
| Tumor necrosis factor | Q9Y5U5 | U | pg/mL | 0.00603 | 77% | 62% | 5.2 |
| receptor superfamily member 18 | | | | | (65%-87%) | (33%-88%) | (1.6-25) |
| Tumor necrosis factor | O95407 | P | pg/ml | 415 | 68% | 86% | 12 |
| receptor superfamily member 6B | | | | | (53%-81%) | (64%-100%) | (3.5-Inf) |
| WNT1-inducible-signaling | O95388 | U | pg/mL | 4.91 | 77% | 54% | 3.9 |
| pathway protein 1 | | | | | (65%-88%) | (27%-80%) | (0.98-19) |

TABLE 1c
Sensitivity, specificity and odds ratio determined by a threshold at the median of the range of analyte
concentrations. RRT treatment discontinuation, if any, starts within 1 day of sample collection.

| | Swiss-Prot | Urine or | | | Sensitivity | Specificity | Odds Ratio |
|---|---|---|---|---|---|---|---|
| Preferred Name | Entry Number | Plasma | Unit | Threshold | (95% CI) | (95% CI) | (95% CI) |
| Antileukoproteinase | P03973 | U | ng/ml | 354 | 48% | 86% | 5.6 |
| | | | | | (27%-70%) | (27%-100%) | (1-Inf) |
| Cathepsin B | P07858 | U | ng/ml | 8.23 | 41% | 92% | 8.2 |
| | | | | | (26%-55%) | (26%-100%) | (1.8-Inf) |
| C-C motif chemokine 1 | P22362 | U | pg/ml | 0.00171 | 67% | 69% | 4.5 |
| | | | | | (53%-79%) | (53%-92%) | (1.5-22) |
| C-C motif chemokine 14 | Q16627 | U | ng/mL | 12.3 | 43% | 92% | 9.1 |
| | | | | | (30%-57%) | (30%-100%) | (2.3-Inf) |
| C-C motif chemokine 16 | O15467 | U | pg/mL | 26.9 | 62% | 69% | 3.7 |
| | | | | | (47%-76%) | (47%-92%) | (1-24) |
| C-C motif chemokine 23 | P55773 | U | pg/mL | 37.2 | 45% | 85% | 4.6 |
| | | | | | (31%-60%) | (31%-100%) | (1.4-Inf) |
| C-C motif chemokine 24 | O00175 | U | pg/ml | 34.4 | 68% | 62% | 3.4 |
| | | | | | (53%-83%) | (53%-87%) | (1.1-17) |
| C-C motif chemokine 28 | Q9NRJ3 | U | pg/mL | 63.8 | 55% | 71% | 3 |
| | | | | | (39%-70%) | (39%-100%) | (0.75-Inf) |
| Chitinase-3-like protein 1 | P36222 | U | ng/ml | 87.1 | 39% | 85% | 3.6 |
| | | | | | (26%-54%) | (26%-100%) | (0.97-Inf) |
| C-X-C motif chemokine 2 | P19875 | U | pg/mL | 56.1 | 44% | 69% | 1.8 |
| | | | | | (30%-59%) | (30%-92%) | (0.54-10) |
| C-X-C motif chemokine 9 | Q07325 | U | pg/ml | 929 | 56% | 77% | 4.3 |
| | | | | | (41%-71%) | (41%-100%) | (1.6-Inf) |
| Dickkopf-related protein 1 | O94907 | U | ng/ml | 1.56 | 38% | 85% | 3.4 |
| | | | | | (24%-52%) | (24%-100%) | (0.87-Inf) |
| Elafin | P19957 | U | ng/ml | 17 | 42% | 100% | >10 |
| | | | | | (21%-66%) | (21%-100%) | |
| Fatty acid-binding protein, | P15090 | P | ng/mL | 215 | 45% | 79% | 3 |
| adipocyte | | | | | (31%-60%) | (31%-100%) | (0.9-Inf) |
| Follistatin-related protein 3 | O95633 | P | ng/mL | 84.8 | 45% | 86% | 4.9 |
| | | | | | (30%-61%) | (30%-100%) | (1.3-Inf) |
| Hepatocyte growth factor-like | P26927 | U | ng/ml | 0.579 | 50% | 77% | 3.3 |
| protein | | | | | (37%-63%) | (37%-100%) | (0.96-Inf) |
| Insulin-like growth factor- | P18065 | U | ng/mL | 234 | 54% | 83% | 5.9 |
| binding protein 2 | | | | | (38%-70%) | (38%-100%) | (1.7-Inf) |
| Insulin-like growth factor- | P22692 | U | ng/mL | 337 | 52% | 77% | 3.5 |
| binding protein 4 | | | | | (35%-68%) | (35%-100%) | (1-Inf) |
| Insulin-like growth factor- | Q16270 | U | ng/ml | 897 | 44% | 62% | 1.3 |
| binding protein 7 | | | | | (30%-59%) | (30%-87%) | (0.33-6.4) |
| Insulin-like growth factor- | Q16270 | P | ng/ml | 897 | 44% | 62% | 1.3 |
| binding protein 7 | | | | | (30%-59%) | (30%-87%) | (0.33-6.4) |
| Kallikrein-14 | Q9P0G3 | U | pg/mL | 23.4 | 55% | 100% | >15 |
| | | | | | (35%-74%) | (35%-100%) | |
| Metalloproteinase inhibitor 1 | P01033 | U | ng/mL | 26.9 | 56% | 92% | 15 |
| | | | | | (43%-69%) | (43%-100%) | (3.8-Inf) |
| Metalloproteinase inhibitor 2 | P16035 | U | ng/mL | 35.4 | 53% | 85% | 6.2 |
| | | | | | (38%-69%) | (38%-100%) | (1.5-Inf) |
| Metalloproteinase inhibitor 4 | Q99727 | U | pg/ml | 61.9 | 52% | 100% | >28 |
| | | | | | (38%-65%) | (38%-100%) | |
| Neutrophil gelatinase- | P80188 | U | ng/mL | 844 | 50% | 85% | 5.5 |
| associated lipocalin | | | | | (36%-64%) | (36%-100%) | (1.5-Inf) |
| Neutrophil gelatinase- | P80188 | P | ng/mL | 844 | 50% | 85% | 5.5 |
| associated lipocalin | | | | | (36%-64%) | (36%-100%) | (1.5-Inf) |
| Nidogen-1 | P14543 | U | ng/ml | 143 | 52% | 77% | 3.5 |
| | | | | | (36%-67%) | (36%-100%) | (1.1-Inf) |
| OX-2 membrane glycoprotein | P41217 | U | pg/mL | 0.0136 | 64% | 85% | 9.6 |
| | | | | | (50%-77%) | (50%-100%) | (3.1-Inf) |
| Pro-interleukin-16 | Q14005 | U | pg/ml | 67.4 | 62% | 85% | 9 |
| | | | | | (46%-77%) | (46%-100%) | (2.5-Inf) |
| Prolactin | P01236 | P | ng/mL | 34.6 | 45% | 86% | 4.9 |
| | | | | | (31%-59%) | (31%-100%) | (1.5-Inf) |
| Renin | P00797 | U | ng/mL | 14.4 | 58% | 85% | 7.7 |
| | | | | | (46%-71%) | (46%-100%) | (2.6-Inf) |
| Tissue factor pathway inhibitor | P10646 | U | pg/ml | 855 | 55% | 86% | 7.2 |
| | | | | | (34%-75%) | (34%-100%) | (1.8-Inf) |
| Tumor necrosis factor | O14763 | P | pg/ml | 404 | 59% | 75% | 4.3 |
| receptor superfamily member 10B | | | | | (40%-75%) | (40%-100%) | (0.86-Inf) |
| Tumor necrosis factor | Q9Y5U5 | U | pg/mL | 0.00841 | 59% | 77% | 4.9 |
| receptor superfamily member 18 | | | | | (47%-72%) | (47%-100%) | (1.8-Inf) |
| Tumor necrosis factor | O95407 | P | pg/ml | 806 | 45% | 93% | 11 |
| receptor superfamily member 6B | | | | | (30%-60%) | (30%-100%) | (2.4-Inf) |
| WNT1-inducible-signaling | O95388 | U | pg/mL | 8.15 | 55% | 85% | 6.8 |
| pathway protein 1 | | | | | (42%-70%) | (42%-100%) | (1.7-Inf) |

TABLE 1d
Sensitivity, specificity and odds ratio determined by a threshold at the 75th percentile of the range of
analyte concentrations. RRT treatment discontinuation, if any, starts within 1 day of sample collection.

| | Swiss-Prot | Urine or | | | Sensitivity | Specificity | Odds Ratio |
|---|---|---|---|---|---|---|---|
| Preferred Name | Entry Number | Plasma | Unit | Threshold | (95% CI) | (95% CI) | (95% CI) |
| Antileukoproteinase | P03973 | U | ng/ml | 525 | 33% | 100% | >3.5 |
| | | | | | (16%-53%) | (100%-100%) | |
| Cathepsin B | P07858 | U | ng/ml | 17.9 | 23% | 100% | >8 |
| | | | | | (10%-38%) | (100%-100%) | |
| C-C motif chemokine 1 | P22362 | U | pg/ml | 9.6 | 29% | 92% | 4.9 |
| | | | | | (15%-44%) | (75%-100%) | (0.95-Inf) |
| C-C motif chemokine 14 | Q16627 | U | ng/mL | 23.1 | 23% | 100% | >7.8 |
| | | | | | (12%-36%) | (100%-100%) | |
| C-C motif chemokine 16 | O15467 | U | pg/mL | 71.1 | 22% | 92% | 3.4 |
| | | | | | (10%-37%) | (75%-100%) | (0.61-Inf) |
| C-C motif chemokine 23 | P55773 | U | pg/mL | 190 | 24% | 100% | >8.3 |
| | | | | | (13%-38%) | (100%-100%) | |
| C-C motif chemokine 24 | O00175 | U | pg/ml | 193 | 26% | 92% | 4.2 |
| | | | | | (15%-38%) | (75%-100%) | (0.94-Inf) |
| C-C motif chemokine 28 | Q9NRJ3 | U | pg/mL | 140 | 21% | 86% | 1.6 |
| | | | | | (7%-38%) | (57%-100%) | (0.3-Inf) |
| Chitinase-3-like protein 1 | P36222 | U | ng/ml | 148 | 21% | 92% | 3.2 |
| | | | | | (11%-33%) | (75%-100%) | (0.64-Inf) |
| C-X-C motif chemokine 2 | P19875 | U | pg/mL | 89.6 | 35% | 100% | >14 |
| | | | | | (22%-49%) | (100%-100%) | |
| C-X-C motif chemokine 9 | Q07325 | U | pg/ml | 16700 | 24% | 92% | 3.8 |
| | | | | | (13%-38%) | (76%-100%) | (0.83-Inf) |
| Dickkopf-related protein 1 | O94907 | U | ng/ml | 2.27 | 24% | 100% | >8.3 |
| | | | | | (13%-37%) | (100%-100%) | |
| Elafin | P19957 | U | ng/ml | 51.1 | 24% | 100% | >4.5 |
| | | | | | (9%-43%) | (100%-100%) | |
| Fatty acid-binding protein, | P15090 | P | ng/mL | 282 | 28% | 86% | 2.4 |
| adipocyte | | | | | (16%-41%) | (64%-100%) | (0.62-Inf) |
| Follistatin-related protein 3 | O95633 | P | ng/mL | 125 | 21% | 100% | >7.2 |
| | | | | | (11%-32%) | (100%-100%) | |
| Hepatocyte growth | P26927 | U | ng/ml | 4.01 | 21% | 100% | >7 |
| factor-like protein | | | | | (10%-34%) | (100%-100%) | |
| Insulin-like growth | P18065 | U | ng/mL | 352 | 35% | 92% | 5.9 |
| factor-binding protein 2 | | | | | (21%-50%) | (75%-100%) | (1.4-Inf) |
| Insulin-like growth | P22692 | U | ng/mL | 679 | 36% | 92% | 6.7 |
| factor-binding protein 4 | | | | | (21%-52%) | (75%-100%) | (1.4-Inf) |
| Insulin-like growth | Q16270 | U | ng/ml | 1650 | 21% | 85% | 1.5 |
| factor-binding protein 7 | | | | | (10%-34%) | (62%-100%) | (0.32-Inf) |
| Insulin-like growth | Q16270 | P | ng/ml | 1650 | 21% | 85% | 1.5 |
| factor-binding protein 7 | | | | | (10%-34%) | (62%-100%) | (0.32-Inf) |
| Kallikrein-14 | Q9P0G3 | U | pg/mL | 40.5 | 35% | 100% | >6.6 |
| | | | | | (18%-56%) | (100%-100%) | |
| Metalloproteinase inhibitor 1 | P01033 | U | ng/mL | 88 | 20% | 100% | >6.4 |
| | | | | | (10%-32%) | (100%-100%) | |
| Metalloproteinase inhibitor 2 | P16035 | U | ng/mL | 76.4 | 26% | 92% | 4.2 |
| | | | | | (14%-39%) | (75%-100%) | (0.84-Inf) |
| Metalloproteinase inhibitor 4 | Q99727 | U | pg/ml | 171 | 20% | 100% | >6.4 |
| | | | | | (9%-32%) | (100%-100%) | |
| Neutrophil gelatinase- | P80188 | U | ng/mL | 1170 | 29% | 92% | 4.9 |
| associated lipocalin | | | | | (18%-41%) | (76%-100%) | (1.2-Inf) |
| Neutrophil gelatinase- | P80188 | P | ng/mL | 1170 | 29% | 92% | 4.9 |
| associated lipocalin | | | | | (18%-41%) | (76%-100%) | (1.2-Inf) |
| Nidogen-1 | P14543 | U | ng/ml | 241 | 29% | 100% | >11 |
| | | | | | (18%-41%) | (100%-100%) | |
| OX-2 membrane glycoprotein | P41217 | U | pg/mL | 15.1 | 38% | 100% | >16 |
| | | | | | (25%-51%) | (100%-100%) | |
| Pro-interleukin-16 | Q14005 | U | pg/ml | 575 | 23% | 100% | >7.6 |
| | | | | | (11%-37%) | (100%-100%) | |
| Prolactin | P01236 | P | ng/mL | 57.2 | 23% | 100% | >8.4 |
| | | | | | (11%-36%) | (100%-100%) | |
| Renin | P00797 | U | ng/mL | 39 | 25% | 100% | >8.5 |
| | | | | | (14%-37%) | (100%-100%) | |
| Tissue factor pathway inhibitor | P10646 | U | pg/ml | 3240 | 24% | 100% | >4.5 |
| | | | | | (7%-44%) | (100%-100%) | |
| Tumor necrosis factor | O14763 | P | pg/ml | 629 | 28% | 100% | >6.3 |
| receptor superfamily member 10B | | | | | (13%-44%) | (100%-100%) | |
| Tumor necrosis factor | Q9Y5U5 | U | pg/mL | 0.012 | 41% | 92% | 8.2 |
| receptor superfamily member 18 | | | | | (28%-54%) | (75%-100%) | (1.9-Inf) |
| Tumor necrosis factor | O95407 | P | pg/ml | 1430 | 22% | 100% | >7.9 |
| receptor superfamily member 6B | | | | | (11%-35%) | (100%-100%) | |
| WNT1-inducible- | O95388 | U | pg/mL | 13.8 | 31% | 92% | 5.3 |
| signaling pathway protein 1 | | | | | (18%-45%) | (77%-100%) | (1.2-Inf) |

TABLE 2a
AUC and the number of “discontinuation”, “not discontinuation” and total
samples where RRT treatment discontinuation, if any, starts within 2 days of sample collection.

| | Swiss-Prot | Urine or | | # | # Not | Total # |
|---|---|---|---|---|---|---|
| Preferred Name | Entry Number | Plasma | AUC (95% CI) | Discontinuation | Discontinuation | samples |
| Antileukoproteinase | P03973 | U | 0.82 (0.66-0.95) | 9 | 31 | 40 |
| Cathepsin B | P07858 | U | 0.75 (0.61-0.87) | 20 | 57 | 77 |
| C-C motif chemokine 1 | P22362 | U | 0.72 (0.57-0.86) | 21 | 58 | 79 |
| C-C motif chemokine 14 | Q16627 | U | 0.77 (0.63-0.88) | 21 | 57 | 78 |
| C-C motif chemokine 16 | O15467 | U | 0.74 (0.60-0.86) | 20 | 56 | 76 |
| C-C motif chemokine 23 | P55773 | U | 0.74 (0.60-0.86) | 21 | 58 | 79 |
| C-C motif chemokine 24 | O00175 | U | 0.71 (0.57-0.84) | 21 | 58 | 79 |
| C-C motif chemokine 28 | Q9NRJ3 | U | 0.77 (0.61-0.92) | 9 | 31 | 40 |
| Chitinase-3-like protein 1 | P36222 | U | 0.76 (0.62-0.88) | 21 | 58 | 79 |
| C-X-C motif chemokine 2 | P19875 | U | 0.76 (0.62-0.88) | 21 | 58 | 79 |
| C-X-C motif chemokine 9 | Q07325 | U | 0.77 (0.62-0.90) | 21 | 58 | 79 |
| Dickkopf-related protein 1 | O94907 | U | 0.74 (0.60-0.86) | 21 | 58 | 79 |
| Elafin | P19957 | U | 0.79 (0.61-0.94) | 9 | 31 | 40 |
| Fatty acid-binding protein, adipocyte | P15090 | P | 0.76 (0.63-0.88) | 23 | 69 | 92 |
| Follistatin-related protein 3 | O95633 | P | 0.74 (0.61-0.87) | 23 | 69 | 92 |
| Hepatocyte growth factor-like protein | P26927 | U | 0.77 (0.64-0.89) | 21 | 58 | 79 |
| Insulin-like growth | P18065 | U | 0.77 (0.60-0.91) | 19 | 56 | 75 |
| factor-binding protein 2 | | | | | | |
| Insulin-like growth | P22692 | U | 0.71 (0.55-0.84) | 20 | 57 | 77 |
| factor-binding protein 4 | | | | | | |
| Insulin-like growth | Q16270 | U | 0.59 (0.45-0.76) | 19 | 53 | 72 |
| factor-binding protein 7 | | | | | | |
| Insulin-like growth | Q16270 | P | 0.73 (0.60-0.85) | 23 | 69 | 92 |
| factor-binding protein 7 | | | | | | |
| Kallikrein-14 | Q9P0G3 | U | 0.85 (0.73-0.97) | 7 | 30 | 37 |
| Metalloproteinase inhibitor 1 | P01033 | U | 0.78 (0.67-0.88) | 21 | 58 | 79 |
| Metalloproteinase inhibitor 2 | P16035 | U | 0.74 (0.58-0.87) | 21 | 58 | 79 |
| Metalloproteinase inhibitor 4 | Q99727 | U | 0.80 (0.68-0.91) | 21 | 58 | 79 |
| Neutrophil gelatinase- | P80188 | U | 0.76 (0.62-0.88) | 21 | 58 | 79 |
| associated lipocalin | | | | | | |
| Neutrophil gelatinase- | P80188 | P | 0.73 (0.58-0.87) | 23 | 69 | 92 |
| associated lipocalin | | | | | | |
| Nidogen-1 | P14543 | U | 0.73 (0.58-0.86) | 21 | 58 | 79 |
| OX-2 membrane glycoprotein | P41217 | U | 0.74 (0.60-0.86) | 21 | 58 | 79 |
| Pro-interleukin-16 | Q14005 | U | 0.73 (0.59-0.86) | 21 | 58 | 79 |
| Prolactin | P01236 | P | 0.77 (0.64-0.88) | 23 | 69 | 92 |
| Renin | P00797 | U | 0.77 (0.64-0.88) | 21 | 57 | 78 |
| Tissue factor pathway inhibitor | P10646 | U | 0.73 (0.57-0.89) | 9 | 31 | 40 |
| Tumor necrosis factor | O14763 | P | 0.75 (0.58-0.93) | 11 | 43 | 54 |
| receptor superfamily member 10B | | | | | | |
| Tumor necrosis factor | Q9Y5U5 | U | 0.72 (0.58-0.86) | 21 | 56 | 77 |
| receptor superfamily member 18 | | | | | | |
| Tumor necrosis factor | O95407 | P | 0.74 (0.60-0.87) | 22 | 69 | 91 |
| receptor superfamily member 6B | | | | | | |
| WNT1-inducible- | O95388 | U | 0.74 (0.57-0.87) | 21 | 57 | 78 |
| signaling pathway protein 1 | | | | | | |

TABLE 2b
Sensitivity, specificity and odds ratio determined by a threshold at the 25th percentile of the range of
analyte concentrations. RRT treatment discontinuation, if any, starts within 2 days of sample collection.

| | Swiss-Prot | Urine or | | | Sensitivity | Specificity | Odds Ratio |
|---|---|---|---|---|---|---|---|
| Preferred Name | Entry Number | Plasma | Unit | Threshold | (95% CI) | (95% CI) | (95% CI) |
| Antileukoproteinase | P03973 | U | ng/ml | 230 | 74% | 67% | 5.8 |
| | | | | | (53%-91%) | (33%-100%) | (1.4-Inf) |
| Cathepsin B | P07858 | U | ng/ml | 1.86 | 74% | 55% | 3.4 |
| | | | | | (60%-86%) | (32%-78%) | (1.3-11) |
| C-C motif chemokine 1 | P22362 | U | pg/ml | 0.0015 | 78% | 52% | 3.8 |
| | | | | | (64%-89%) | (30%-75%) | (1.3-14) |
| C-C motif chemokine 14 | Q16627 | U | ng/mL | 5.73 | 77% | 57% | 4.5 |
| | | | | | (64%-89%) | (33%-80%) | (1.4-17) |
| C-C motif chemokine 16 | O15467 | U | pg/mL | 15.2 | 84% | 55% | 6.4 |
| | | | | | (72%-94%) | (33%-76%) | (2.2-25) |
| C-C motif chemokine 23 | P55773 | U | pg/mL | 7.68 | 88% | 43% | 5.5 |
| | | | | | (77%-96%) | (18%-67%) | (1.5-24) |
| C-C motif chemokine 24 | O00175 | U | pg/ml | 32.1 | 72% | 52% | 2.9 |
| | | | | | (56%-88%) | (30%-75%) | (0.94-11) |
| C-C motif chemokine 28 | Q9NRJ3 | U | pg/mL | 28.3 | 81% | 56% | 5.2 |
| | | | | | (66%-93%) | (38%-80%) | (2.1-22) |
| Chitinase-3-like protein 1 | P36222 | U | ng/ml | 15.8 | 79% | 67% | 7.7 |
| | | | | | (67%-90%) | (43%-88%) | (2.7-33) |
| C-X-C motif chemokine 2 | P19875 | U | pg/mL | 26.8 | 76% | 67% | 6.3 |
| | | | | | (64%-87%) | (45%-86%) | (2.2-25) |
| C-X-C motif chemokine 9 | Q07325 | U | pg/ml | 208 | 84% | 52% | 6 |
| | | | | | (73%-95%) | (28%-75%) | (1.8-27) |
| Dickkopf-related protein 1 | O94907 | U | ng/ml | 0.294 | 79% | 48% | 3.5 |
| | | | | | (67%-92%) | (24%-71%) | (0.98-14) |
| Elafin | P19957 | U | ng/ml | 8.44 | 65% | 67% | 3.6 |
| | | | | | (43%-84%) | (30%-100%) | (0.74-Inf) |
| Fatty acid-binding protein, | P15090 | P | ng/mL | 119 | 75% | 52% | 3.3 |
| adipocyte | | | | | (63%-87%) | (34%-74%) | (1.3-11) |
| Follistatin-related protein 3 | O95633 | P | ng/mL | 48.2 | 80% | 39% | 2.5 |
| | | | | | (66%-92%) | (20%-63%) | (0.83-10) |
| Hepatocyte growth factor-like | P26927 | U | ng/ml | 0.219 | 84% | 67% | 11 |
| protein | | | | | (73%-95%) | (47%-85%) | (4.1-48) |
| Insulin-like growth factor- | P18065 | U | ng/mL | 97.9 | 80% | 58% | 5.6 |
| binding protein 2 | | | | | (67%-92%) | (33%-82%) | (1.7-25) |
| Insulin-like growth factor- | P22692 | U | ng/mL | 135 | 79% | 45% | 3.1 |
| binding protein 4 | | | | | (66%-91%) | (22%-68%) | (0.93-11) |
| Insulin-like growth factor- | Q16270 | U | ng/ml | 418 | 74% | 48% | 2.6 |
| binding protein 7 | | | | | (58%-89%) | (26%-68%) | (0.78-9.6) |
| Insulin-like growth factor- | Q16270 | P | ng/ml | 131 | 74% | 48% | 2.6 |
| binding protein 7 | | | | | (58%-89%) | (26%-68%) | (0.78-9.6) |
| Kallikrein-14 | Q9P0G3 | U | pg/mL | 12.6 | 73% | 71% | 6.9 |
| | | | | | (61%-86%) | (40%-100%) | (1.4-Inf) |
| Metalloproteinase inhibitor 1 | P01033 | U | ng/mL | 15.4 | 81% | 57% | 5.7 |
| | | | | | (70%-91%) | (38%-76%) | (2.1-18) |
| Metalloproteinase inhibitor 2 | P16035 | U | ng/mL | 18.6 | 78% | 57% | 4.6 |
| | | | | | (64%-90%) | (36%-77%) | (1.6-18) |
| Metalloproteinase inhibitor 4 | Q99727 | U | pg/ml | 29.9 | 83% | 57% | 6.4 |
| | | | | | (72%-92%) | (35%-80%) | (2.3-24) |
| Neutrophil gelatinase- | P80188 | U | ng/mL | 468 | 79% | 57% | 5.1 |
| associated lipocalin | | | | | (67%-90%) | (33%-80%) | (1.6-21) |
| Neutrophil gelatinase- | P80188 | P | ng/mL | 440 | 79% | 57% | 5.1 |
| associated lipocalin | | | | | (67%-90%) | (33%-80%) | (1.6-21) |
| Nidogen-1 | P14543 | U | ng/ml | 84.8 | 84% | 62% | 8.8 |
| | | | | | (74%-93%) | (38%-84%) | (2.8-38) |
| OX-2 membrane glycoprotein | P41217 | U | pg/mL | 0.00995 | 72% | 52% | 2.9 |
| | | | | | (58%-85%) | (30%-76%) | (1-10) |
| Pro-interleukin-16 | Q14005 | U | pg/ml | 0.0289 | 84% | 43% | 4.1 |
| | | | | | (74%-94%) | (21%-64%) | (1.2-16) |
| Prolactin | P01236 | P | ng/mL | 16.1 | 86% | 52% | 6.4 |
| | | | | | (77%-93%) | (29%-75%) | (2.4-21) |
| Renin | P00797 | U | ng/mL | 6.71 | 77% | 52% | 3.7 |
| | | | | | (65%-89%) | (28%-76%) | (1.3-12) |
| Tissue factor pathway inhibitor | P10646 | U | pg/ml | 315 | 74% | 33% | 1.4 |
| | | | | | (54%-91%) | (8%-71%) | (0.21-12) |
| Tumor necrosis factor | O14763 | P | pg/ml | 289 | 81% | 64% | 7.7 |
| receptor superfamily member 10B | | | | | (65%-95%) | (33%-100%) | (1.8-Inf) |
| Tumor necrosis factor | Q9Y5U5 | U | pg/mL | 0.00514 | 77% | 48% | 3 |
| receptor superfamily member 18 | | | | | (64%-88%) | (25%-70%) | (0.91-11) |
| Tumor necrosis factor | O95407 | P | pg/ml | 267 | 75% | 32% | 1.4 |
| receptor superfamily member 6B | | | | | (61%-88%) | (14%-53%) | (0.4-5) |
| WNT1-inducible-signaling | O95388 | U | pg/mL | 4.14 | 88% | 48% | 6.5 |
| pathway protein 1 | | | | | (78%-96%) | (26%-70%) | (2.1-27) |

TABLE 2c
Sensitivity, specificity and odds ratio determined by a threshold at the median of the range of analyte
concentrations. RRT treatment discontinuation, if any, starts within 2 days of sample collection.

| | Swiss-Prot | Urine or | | | Sensitivity | Specificity | Odds Ratio |
|---|---|---|---|---|---|---|---|
| Preferred Name | Entry Number | Plasma | Unit | Threshold | (95% CI) | (95% CI) | (95% CI) |
| Antileukoproteinase | P03973 | U | ng/ml | 342 | 55% | 78% | 4.3 |
| | | | | | (32%-76%) | (32%-100%) | (0.75-Inf) |
| Cathepsin B | P07858 | U | ng/ml | 3.11 | 63% | 75% | 5.1 |
| | | | | | (47%-78%) | (47%-95%) | (1.7-35) |
| C-C motif chemokine 1 | P22362 | U | pg/ml | 1.14 | 59% | 76% | 4.5 |
| | | | | | (42%-74%) | (42%-95%) | (1.4-30) |
| C-C motif chemokine 14 | Q16627 | U | ng/mL | 8.73 | 56% | 76% | 4.1 |
| | | | | | (41%-71%) | (41%-93%) | (1.4-18) |
| C-C motif chemokine 16 | O15467 | U | pg/mL | 27.9 | 59% | 80% | 5.7 |
| | | | | | (41%-76%) | (41%-95%) | (1.8-36) |
| C-C motif chemokine 23 | P55773 | U | pg/mL | 26.3 | 62% | 67% | 3.3 |
| | | | | | (46%-77%) | (46%-88%) | (1.1-13) |
| C-C motif chemokine 24 | O00175 | U | pg/ml | 78.9 | 59% | 81% | 6 |
| | | | | | (43%-75%) | (43%-95%) | (2.1-34) |
| C-C motif chemokine 28 | Q9NRJ3 | U | pg/mL | 54.6 | 61% | 78% | 5.5 |
| | | | | | (44%-77%) | (44%-100%) | (1.3-Inf) |
| Chitinase-3-like protein 1 | P36222 | U | ng/ml | 49.6 | 60% | 81% | 6.5 |
| | | | | | (46%-75%) | (46%-96%) | (2.2-36) |
| C-X-C motif chemokine 2 | P19875 | U | pg/mL | 43.8 | 59% | 71% | 3.5 |
| | | | | | (43%-74%) | (43%-89%) | (1.3-14) |
| C-X-C motif chemokine 9 | Q07325 | U | pg/ml | 1070 | 59% | 76% | 4.5 |
| | | | | | (42%-74%) | (42%-95%) | (1.5-30) |
| Dickkopf-related protein 1 | O94907 | U | ng/ml | 0.717 | 60% | 67% | 3 |
| | | | | | (46%-75%) | (46%-88%) | (1.1-12) |
| Elafin | P19957 | U | ng/ml | 12.5 | 55% | 89% | 9.7 |
| | | | | | (30%-79%) | (30%-100%) | (1.6-Inf) |
| Fatty acid-binding protein, | P15090 | P | ng/mL | 174 | 61% | 74% | 4.4 |
| adipocyte | | | | | (45%-77%) | (45%-95%) | (1.4-32) |
| Follistatin-related protein 3 | O95633 | P | ng/mL | 77.9 | 58% | 87% | 9.2 |
| | | | | | (42%-74%) | (42%-100%) | (3-Inf) |
| Hepatocyte growth factor-like | P26927 | U | ng/ml | 0.485 | 60% | 76% | 4.9 |
| protein | | | | | (46%-75%) | (46%-93%) | (1.9-20) |
| Insulin-like growth factor- | P18065 | U | ng/mL | 274 | 48% | 79% | 3.5 |
| binding protein 2 | | | | | (33%-63%) | (33%-100%) | (1.1-Inf) |
| Insulin-like growth factor- | P22692 | U | ng/mL | 255 | 63% | 65% | 3.2 |
| binding protein 4 | | | | | (47%-78%) | (47%-88%) | (1-13) |
| Insulin-like growth factor- | Q16270 | U | ng/ml | 779 | 57% | 67% | 2.6 |
| binding protein 7 | | | | | (40%-73%) | (40%-86%) | (0.88-9.7) |
| Insulin-like growth factor- | Q16270 | P | ng/ml | 779 | 57% | 67% | 2.6 |
| binding protein 7 | | | | | (40%-73%) | (40%-86%) | (0.88-9.7) |
| Kallikrein-14 | Q9P0G3 | U | pg/mL | 23.7 | 53% | 100% | >14 |
| | | | | | (35%-72%) | (35%-100%) | |
| Metalloproteinase inhibitor 1 | P01033 | U | ng/mL | 26.7 | 60% | 81% | 6.5 |
| | | | | | (46%-74%) | (46%-95%) | (2.4-33) |
| Metalloproteinase inhibitor 2 | P16035 | U | ng/mL | 37.2 | 57% | 81% | 5.6 |
| | | | | | (41%-74%) | (41%-95%) | (1.8-35) |
| Metalloproteinase inhibitor 4 | Q99727 | U | pg/ml | 56.7 | 60% | 86% | 9.1 |
| | | | | | (46%-74%) | (46%-100%) | (3.3-Inf) |
| Neutrophil gelatinase- | P80188 | U | ng/mL | 737 | 59% | 76% | 4.5 |
| associated lipocalin | | | | | (44%-73%) | (44%-94%) | (1.5-22) |
| Neutrophil gelatinase- | P80188 | P | ng/mL | 737 | 59% | 76% | 4.5 |
| associated lipocalin | | | | | (44%-73%) | (44%-94%) | (1.5-22) |
| Nidogen-1 | P14543 | U | ng/ml | 144 | 53% | 71% | 2.9 |
| | | | | | (38%-69%) | (38%-92%) | (0.94-14) |
| OX-2 membrane glycoprotein | P41217 | U | pg/mL | 2.1 | 57% | 86% | 7.9 |
| | | | | | (42%-71%) | (42%-100%) | (2.9-Inf) |
| Pro-interleukin-16 | Q14005 | U | pg/ml | 133 | 59% | 81% | 6 |
| | | | | | (42%-74%) | (42%-100%) | (1.9-Inf) |
| Prolactin | P01236 | P | ng/mL | 31.8 | 55% | 78% | 4.4 |
| | | | | | (41%-69%) | (41%-94%) | (1.5-22) |
| Renin | P00797 | U | ng/mL | 15.2 | 56% | 81% | 5.4 |
| | | | | | (43%-70%) | (43%-96%) | (1.9-31) |
| Tissue factor pathway | P10646 | U | pg/ml | 764 | 61% | 89% | 13 |
| inhibitor | | | | | (41%-81%) | (41%-100%) | (3.1-Inf) |
| Tumor necrosis factor | O14763 | P | pg/ml | 414 | 58% | 73% | 3.7 |
| receptor superfamily member 10B | | | | | (39%-74%) | (39%-100%) | (1.2-Inf) |
| Tumor necrosis factor | Q9Y5U5 | U | pg/mL | 0.00936 | 52% | 76% | 3.4 |
| receptor superfamily member 18 | | | | | (39%-64%) | (39%-91%) | (1.3-14) |
| Tumor necrosis factor | O95407 | P | pg/ml | 690 | 55% | 91% | 12 |
| receptor superfamily member 6B | | | | | (39%-70%) | (39%-100%) | (2.8-Inf) |
| WNT1-inducible-signaling | O95388 | U | pg/mL | 8.32 | 58% | 81% | 5.8 |
| pathway protein 1 | | | | | (43%-74%) | (43%-96%) | (1.8-38) |

TABLE 2d
Sensitivity, specificity and odds ratio determined by a threshold at the 75th percentile of the range of
analyte concentrations. RRT treatment discontinuation, if any, starts within 2 days of sample collection.

| | Swiss-Prot | Urine or | | | Sensitivity | Specificity | Odds Ratio |
|---|---|---|---|---|---|---|---|
| Preferred Name | Entry Number | Plasma | Unit | Threshold | (95% CI) | (95% CI) | (95% CI) |
| Antileukoproteinase | P03973 | U | ng/ml | 695 | 23% | 100% | >2 |
| | | | | | (9%-39%) | (100%-100%) | |
| Cathepsin B | P07858 | U | ng/ml | 12.7 | 33% | 95% | 9.5 |
| | | | | | (19%-48%) | (83%-100%) | (2-Inf) |
| C-C motif chemokine 1 | P22362 | U | pg/ml | 10.4 | 31% | 90% | 4.3 |
| | | | | | (16%-47%) | (77%-100%) | (1.1-Inf) |
| C-C motif chemokine 14 | Q16627 | U | ng/mL | 18.2 | 35% | 95% | 11 |
| | | | | | (22%-49%) | (84%-100%) | (2.4-Inf) |
| C-C motif chemokine 16 | O15467 | U | pg/mL | 44 | 39% | 90% | 5.8 |
| | | | | | (24%-55%) | (75%-100%) | (1.6-Inf) |
| C-C motif chemokine 23 | P55773 | U | pg/mL | 101 | 34% | 95% | 11 |
| | | | | | (20%-50%) | (84%-100%) | (2.3-Inf) |
| C-C motif chemokine 24 | O00175 | U | pg/ml | 145 | 38% | 95% | 12 |
| | | | | | (24%-53%) | (84%-100%) | (2.8-Inf) |
| C-C motif chemokine 28 | Q9NRJ3 | U | pg/mL | 114 | 29% | 89% | 3.3 |
| | | | | | (12%-50%) | (63%-100%) | (0.56-Inf) |
| Chitinase-3-like protein 1 | P36222 | U | ng/ml | 104 | 36% | 86% | 3.4 |
| | | | | | (21%-52%) | (70%-100%) | (0.98-Inf) |
| C-X-C motif chemokine 2 | P19875 | U | pg/mL | 152 | 28% | 95% | 7.6 |
| | | | | | (14%-42%) | (84%-100%) | (1.6-Inf) |
| C-X-C motif chemokine 9 | Q07325 | U | pg/ml | 12000 | 31% | 90% | 4.3 |
| | | | | | (17%-46%) | (75%-100%) | (1.1-Inf) |
| Dickkopf-related protein 1 | O94907 | U | ng/ml | 1.56 | 41% | 90% | 6.7 |
| | | | | | (27%-58%) | (75%-100%) | (1.8-Inf) |
| Elafin | P19957 | U | ng/ml | 39.4 | 32% | 100% | >6.7 |
| | | | | | (13%-53%) | (100%-100%) | |
| Fatty acid-binding protein, | P15090 | P | ng/mL | 311 | 28% | 91% | 4 |
| adipocyte | | | | | (15%-41%) | (79%-100%) | (1.1-Inf) |
| Follistatin-related protein 3 | O95633 | P | ng/mL | 109 | 32% | 100% | >13 |
| | | | | | (19%-46%) | (100%-100%) | |
| Hepatocyte growth | P26927 | U | ng/ml | 2.13 | 34% | 95% | 11 |
| factor-like protein | | | | | (21%-49%) | (84%-100%) | (2.3-Inf) |
| Insulin-like growth | P18065 | U | ng/mL | 524 | 32% | 95% | 8.5 |
| factor-binding protein 2 | | | | | (17%-49%) | (82%-100%) | (1.8-Inf) |
| Insulin-like growth | P22692 | U | ng/mL | 1090 | 26% | 90% | 3.2 |
| factor-binding protein 4 | | | | | (14%-40%) | (75%-100%) | (0.88-Inf) |
| Insulin-like growth | Q16270 | U | ng/ml | 1690 | 21% | 86% | 1.6 |
| factor-binding protein 7 | | | | | (9%-35%) | (68%-100%) | (0.39-Inf) |
| Insulin-like growth | Q16270 | P | ng/ml | 1690 | 21% | 86% | 1.6 |
| factor-binding protein 7 | | | | | (9%-35%) | (68%-100%) | (0.39-Inf) |
| Kallikrein-14 | Q9P0G3 | U | pg/mL | 50.3 | 30% | 100% | >5.1 |
| | | | | | (13%-50%) | (100%-100%) | |
| Metalloproteinase inhibitor 1 | P01033 | U | ng/mL | 49.5 | 40% | 100% | >17 |
| | | | | | (25%-55%) | (100%-100%) | |
| Metalloproteinase inhibitor 2 | P16035 | U | ng/mL | 75.1 | 29% | 86% | 2.5 |
| | | | | | (16%-45%) | (68%-100%) | (0.72-Inf) |
| Metalloproteinase inhibitor 4 | Q99727 | U | pg/ml | 82.1 | 43% | 95% | 15 |
| | | | | | (28%-59%) | (83%-100%) | (3.3-Inf) |
| Neutrophil gelatinase- | P80188 | U | ng/mL | 1160 | 31% | 86% | 2.7 |
| associated lipocalin | | | | | (19%-43%) | (68%-100%) | (0.83-Inf) |
| Neutrophil gelatinase- | P80188 | P | ng/mL | 1160 | 31% | 86% | 2.7 |
| associated lipocalin | | | | | (19%-43%) | (68%-100%) | (0.83-Inf) |
| Nidogen-1 | P14543 | U | ng/ml | 215 | 38% | 90% | 5.8 |
| | | | | | (24%-53%) | (76%-100%) | (1.6-Inf) |
| OX-2 membrane glycoprotein | P41217 | U | pg/mL | 37 | 33% | 95% | 9.7 |
| | | | | | (20%-46%) | (84%-100%) | (2.1-Inf) |
| Pro-interleukin-16 | Q14005 | U | pg/ml | 525 | 31% | 95% | 9 |
| | | | | | (16%-47%) | (86%-100%) | (2.2-Inf) |
| Prolactin | P01236 | P | ng/mL | 41.8 | 39% | 91% | 6.8 |
| | | | | | (26%-53%) | (77%-100%) | (1.9-Inf) |
| Renin | P00797 | U | ng/mL | 29.1 | 37% | 90% | 5.5 |
| | | | | | (22%-53%) | (75%-100%) | (1.5-Inf) |
| Tissue factor pathway inhibitor | P10646 | U | pg/ml | 1910 | 35% | 100% | >7.7 |
| | | | | | (16%-57%) | (100%-100%) | |
| Tumor necrosis factor | O14763 | P | pg/ml | 530 | 40% | 91% | 6.5 |
| receptor superfamily member 10B | | | | | (20%-59%) | (67%-100%) | (1-Inf) |
| Tumor necrosis factor | Q9Y5U5 | U | pg/mL | 1.54 | 27% | 95% | 7.3 |
| receptor superfamily member 18 | | | | | (14%-41%) | (86%-100%) | (1.7-Inf) |
| Tumor necrosis factor | O95407 | P | pg/ml | 1150 | 32% | 91% | 4.7 |
| receptor superfamily member 6B | | | | | (19%-46%) | (72%-100%) | (1.1-Inf) |
| WNT1-inducible- | O95388 | U | pg/mL | 14.3 | 28% | 86% | 2.3 |
| signaling pathway protein 1 | | | | | (16%-42%) | (68%-100%) | (0.67-Inf) |

TABLE 3a
AUC and the number of “discontinuation”, “not discontinuation” and total
samples where RRT treatment discontinuation, if any, starts within 3 days of sample collection.

| | Swiss-Prot | Urine or | | # | # Not | Total # |
|---|---|---|---|---|---|---|
| Preferred Name | Entry Number | Plasma | AUC (95% CI) | Discontinuation | Discontinuation | samples |
| Antileukoproteinase | P03973 | U | 0.79 (0.62-0.94) | 12 | 28 | 40 |
| Cathepsin B | P07858 | U | 0.79 (0.66-0.90) | 28 | 49 | 77 |
| C-C motif chemokine 1 | P22362 | U | 0.73 (0.59-0.85) | 29 | 50 | 79 |
| C-C motif chemokine 14 | Q16627 | U | 0.81 (0.69-0.91) | 29 | 49 | 78 |
| C-C motif chemokine 16 | O15467 | U | 0.74 (0.58-0.87) | 28 | 48 | 76 |
| C-C motif chemokine 23 | P55773 | U | 0.79 (0.64-0.91) | 29 | 50 | 79 |
| C-C motif chemokine 24 | O00175 | U | 0.71 (0.57-0.86) | 29 | 50 | 79 |
| C-C motif chemokine 28 | Q9NRJ3 | U | 0.73 (0.52-0.94) | 12 | 28 | 40 |
| Chitinase-3-like protein 1 | P36222 | U | 0.79 (0.65-0.90) | 29 | 50 | 79 |
| C-X-C motif chemokine 2 | P19875 | U | 0.75 (0.62-0.87) | 29 | 50 | 79 |
| C-X-C motif chemokine 9 | Q07325 | U | 0.79 (0.66-0.90) | 29 | 50 | 79 |
| Dickkopf-related protein 1 | O94907 | U | 0.76 (0.62-0.88) | 29 | 50 | 79 |
| Elafin | P19957 | U | 0.79 (0.63-0.92) | 12 | 28 | 40 |
| Fatty acid-binding protein, | P15090 | P | 0.75 (0.61-0.86) | 33 | 59 | 92 |
| adipocyte | | | | | | |
| Follistatin-related protein 3 | O95633 | P | 0.74 (0.60-0.87) | 33 | 59 | 92 |
| Hepatocyte growth factor- | P26927 | U | 0.81 (0.68-0.92) | 29 | 50 | 79 |
| like protein | | | | | | |
| Insulin-like growth factor- | P18065 | U | 0.76 (0.62-0.89) | 27 | 48 | 75 |
| binding protein 2 | | | | | | |
| Insulin-like growth factor- | P22692 | U | 0.74 (0.59-0.86) | 28 | 49 | 77 |
| binding protein 4 | | | | | | |
| Insulin-like growth factor- | Q16270 | U | 0.55 (0.41-0.73) | 26 | 46 | 72 |
| binding protein 7 | | | | | | |
| Insulin-like growth factor- | Q16270 | P | 0.74 (0.60-0.86) | 33 | 59 | 92 |
| binding protein 7 | | | | | | |
| Kallikrein-14 | Q9P0G3 | U | 0.83 (0.64-0.98) | 10 | 27 | 37 |
| Metalloproteinase inhibitor 1 | P01033 | U | 0.76 (0.63-0.88) | 29 | 50 | 79 |
| Metalloproteinase inhibitor 2 | P16035 | U | 0.70 (0.56-0.84) | 29 | 50 | 79 |
| Metalloproteinase inhibitor 4 | Q99727 | U | 0.82 (0.70-0.92) | 29 | 50 | 79 |
| Neutrophil gelatinase- | P80188 | U | 0.77 (0.64-0.87) | 29 | 50 | 79 |
| associated lipocalin | | | | | | |
| Neutrophil gelatinase- | P80188 | P | 0.74 (0.61-0.85) | 33 | 59 | 92 |
| associated lipocalin | | | | | | |
| Nidogen-1 | P14543 | U | 0.75 (0.61-0.88) | 29 | 50 | 79 |
| OX-2 membrane glycoprotein | P41217 | U | 0.74 (0.60-0.86) | 29 | 50 | 79 |
| Pro-interleukin-16 | Q14005 | U | 0.72 (0.57-0.85) | 29 | 50 | 79 |
| Prolactin | P01236 | P | 0.76 (0.63-0.86) | 33 | 59 | 92 |
| Renin | P00797 | U | 0.76 (0.62-0.87) | 29 | 49 | 78 |
| Tissue factor pathway inhibitor | P10646 | U | 0.77 (0.62-0.93) | 12 | 28 | 40 |
| Tumor necrosis factor | O14763 | P | 0.73 (0.55-0.88) | 15 | 39 | 54 |
| receptor superfamily member 10B | | | | | | |
| Tumor necrosis factor | Q9Y5U5 | U | 0.72 (0.61-0.83) | 29 | 48 | 77 |
| receptor superfamily member 18 | | | | | | |
| Tumor necrosis factor | O95407 | P | 0.76 (0.64-0.87) | 32 | 59 | 91 |
| receptor superfamily member 6B | | | | | | |
| WNT1-inducible-signaling | O95388 | U | 0.73 (0.58-0.86) | 29 | 49 | 78 |
| pathway protein 1 | | | | | | |

TABLE 3b
Sensitivity, specificity and odds ratio determined by a threshold at the 25th percentile of the range of
analyte concentrations. RRT treatment discontinuation, if any, starts within 3 days of sample collection.

| | Swiss-Prot | Urine or | | | Sensitivity | Specificity | Odds Ratio |
|---|---|---|---|---|---|---|---|
| Preferred Name | Entry Number | Plasma | Unit | Threshold | (95% CI) | (95% CI) | (95% CI) |
| Antileukoproteinase | P03973 | U | ng/ml | 129 | 89% | 42% | 6 |
| | | | | | (76%-100%) | (20%-67%) | (1.6-Inf) |
| Cathepsin B | P07858 | U | ng/ml | 0.713 | 90% | 21% | 2.4 |
| | | | | | (79%-98%) | (4%-42%) | (0.3-18) |
| C-C motif chemokine 1 | P22362 | U | pg/ml | 0.0016 | 78% | 52% | 3.8 |
| | | | | | (63%-90%) | (35%-68%) | (1.4-12) |
| C-C motif chemokine 14 | Q16627 | U | ng/mL | 2.66 | 96% | 38% | 14 |
| | | | | | (90%-100%) | (19%-57%) | (3.5-Inf) |
| C-C motif chemokine 16 | O15467 | U | pg/mL | 16.2 | 83% | 46% | 4.3 |
| | | | | | (70%-95%) | (25%-68%) | (1.3-22) |
| C-C motif chemokine 23 | P55773 | U | pg/mL | 8.8 | 92% | 52% | 12 |
| | | | | | (82%-100%) | (29%-73%) | (3.3-Inf) |
| C-C motif chemokine 24 | O00175 | U | pg/ml | 13.2 | 86% | 31% | 2.8 |
| | | | | | (71%-98%) | (12%-54%) | (0.62-25) |
| C-C motif chemokine 28 | Q9NRJ3 | U | pg/mL | 14.8 | 89% | 50% | 8.3 |
| | | | | | (76%-100%) | (33%-83%) | (2.5-Inf) |
| Chitinase-3-like protein 1 | P36222 | U | ng/ml | 10.6 | 88% | 41% | 5.2 |
| | | | | | (76%-97%) | (19%-64%) | (1.3-30) |
| C-X-C motif chemokine 2 | P19875 | U | pg/mL | 16.3 | 90% | 41% | 6.4 |
| | | | | | (79%-98%) | (21%-62%) | (1.8-43) |
| C-X-C motif chemokine 9 | Q07325 | U | pg/ml | 118 | 96% | 31% | 11 |
| | | | | | (87%-100%) | (13%-48%) | (2-Inf) |
| Dickkopf-related protein 1 | O94907 | U | ng/ml | 0.272 | 82% | 38% | 2.8 |
| | | | | | (67%-95%) | (19%-58%) | (0.8-14) |
| Elafin | P19957 | U | ng/ml | 5.42 | 96% | 50% | 27 |
| | | | | | (88%-100%) | (14%-80%) | (3.1-Inf) |
| Fatty acid-binding protein, | P15090 | P | ng/mL | 79.2 | 95% | 39% | 12 |
| adipocyte | | | | | (89%-100%) | (22%-57%) | (3.6-Inf) |
| Follistatin-related protein 3 | O95633 | P | ng/mL | 47.8 | 83% | 36% | 2.8 |
| | | | | | (69%-95%) | (18%-57%) | (0.85-14) |
| Hepatocyte growth factor-like | P26927 | U | ng/ml | 0.145 | 92% | 38% | 7 |
| protein | | | | | (82%-100%) | (18%-60%) | (1.8-Inf) |
| Insulin-like growth factor- | P18065 | U | ng/mL | 77.4 | 90% | 41% | 5.9 |
| binding protein 2 | | | | | (78%-100%) | (19%-64%) | (1.4-Inf) |
| Insulin-like growth factor- | P22692 | U | ng/mL | 115 | 88% | 43% | 5.4 |
| binding protein 4 | | | | | (76%-97%) | (21%-65%) | (1.5-30) |
| Insulin-like growth factor- | Q16270 | U | ng/ml | 346 | 80% | 31% | 1.8 |
| binding protein 7 | | | | | (65%-94%) | (17%-46%) | (0.58-7.6) |
| Insulin-like growth factor- | Q16270 | P | ng/ml | 117 | 80% | 31% | 1.8 |
| binding protein 7 | | | | | (65%-94%) | (17%-46%) | (0.58-7.6) |
| Kallikrein-14 | Q9P0G3 | U | pg/mL | 4.96 | 96% | 40% | 17 |
| | | | | | (88%-100%) | (13%-83%) | (2.6-Inf) |
| Metalloproteinase inhibitor 1 | P01033 | U | ng/mL | 11.6 | 82% | 34% | 2.4 |
| | | | | | (69%-92%) | (17%-55%) | (0.69-9.6) |
| Metalloproteinase inhibitor 2 | P16035 | U | ng/mL | 15.1 | 88% | 38% | 4.5 |
| | | | | | (80%-96%) | (19%-59%) | (1.4-18) |
| Metalloproteinase inhibitor 4 | Q99727 | U | pg/ml | 24.8 | 92% | 41% | 8.1 |
| | | | | | (81%-100%) | (20%-64%) | (2-Inf) |
| Neutrophil gelatinase- | P80188 | U | ng/mL | 326 | 94% | 52% | 17 |
| associated lipocalin | | | | | (87%-100%) | (31%-71%) | (5.5-Inf) |
| Neutrophil gelatinase- | P80188 | P | ng/mL | 456 | 94% | 52% | 17 |
| associated lipocalin | | | | | (87%-100%) | (31%-71%) | (5.5-Inf) |
| Nidogen-1 | P14543 | U | ng/ml | 77.3 | 88% | 48% | 6.8 |
| | | | | | (77%-97%) | (27%-70%) | (2-42) |
| OX-2 membrane glycoprotein | P41217 | U | pg/mL | 0.00716 | 88% | 31% | 3.3 |
| | | | | | (77%-96%) | (15%-48%) | (0.99-15) |
| Pro-interleukin-16 | Q14005 | U | pg/ml | 0.0397 | 76% | 48% | 3 |
| | | | | | (60%-90%) | (28%-68%) | (0.92-11) |
| Prolactin | P01236 | P | ng/mL | 15.5 | 92% | 42% | 8 |
| | | | | | (84%-98%) | (23%-62%) | (2.4-37) |
| Renin | P00797 | U | ng/mL | 3.81 | 94% | 28% | 5.8 |
| | | | | | (88%-100%) | (12%-45%) | (1.6-Inf) |
| Tissue factor pathway inhibitor | P10646 | U | pg/ml | 336 | 75% | 42% | 2.1 |
| | | | | | (57%-90%) | (17%-78%) | (0.5-16) |
| Tumor necrosis factor | O14763 | P | pg/ml | 251 | 87% | 33% | 3.4 |
| receptor superfamily member 10B | | | | | (71%-100%) | (8%-65%) | (0.42-Inf) |
| Tumor necrosis factor | Q9Y5U5 | U | pg/mL | 0.00453 | 88% | 34% | 3.7 |
| receptor superfamily member 18 | | | | | (75%-98%) | (17%-55%) | (1.1-26) |
| Tumor necrosis factor | O95407 | P | pg/ml | 176 | 83% | 25% | 1.6 |
| receptor superfamily member 6B | | | | | (69%-95%) | (10%-43%) | (0.45-7) |
| WNT1-inducible-signaling | O95388 | U | pg/mL | 5.09 | 84% | 48% | 4.8 |
| pathway protein 1 | | | | | (73%-92%) | (29%-69%) | (1.6-18) |

TABLE 3c
Sensitivity, specificity and odds ratio determined by a threshold at the median of the range of analyte
concentrations. RRT treatment discontinuation, if any, starts within 3 days of sample collection.

| | Swiss-Prot | Urine or | | | Sensitivity | Specificity | Odds Ratio |
|---|---|---|---|---|---|---|---|
| Preferred Name | Entry Number | Plasma | Unit | Threshold | (95% CI) | (95% CI) | (95% CI) |
| Antileukoproteinase | P03973 | U | ng/ml | 277 | 75% | 75% | 9 |
| | | | | | (57%-90%) | (57%-100%) | (2.1-Inf) |
| Cathepsin B | P07858 | U | ng/ml | 3.4 | 71% | 82% | 12 |
| | | | | | (54%-87%) | (54%-94%) | (3.9-58) |
| C-C motif chemokine 1 | P22362 | U | pg/ml | 2.25 | 56% | 79% | 4.9 |
| | | | | | (38%-72%) | (38%-94%) | (1.6-23) |
| C-C motif chemokine 14 | Q16627 | U | ng/mL | 6.66 | 78% | 62% | 5.7 |
| | | | | | (63%-91%) | (63%-81%) | (1.9-23) |
| C-C motif chemokine 16 | O15467 | U | pg/mL | 31.7 | 56% | 82% | 5.9 |
| | | | | | (37%-74%) | (37%-95%) | (1.9-30) |
| C-C motif chemokine 23 | P55773 | U | pg/mL | 26.5 | 68% | 72% | 5.6 |
| | | | | | (50%-84%) | (50%-91%) | (1.8-28) |
| C-C motif chemokine 24 | O00175 | U | pg/ml | 67.4 | 62% | 72% | 4.3 |
| | | | | | (45%-80%) | (45%-89%) | (1.4-19) |
| C-C motif chemokine 28 | Q9NRJ3 | U | pg/mL | 66.4 | 57% | 75% | 4 |
| | | | | | (40%-74%) | (40%-100%) | (1-Inf) |
| Chitinase-3-like protein 1 | P36222 | U | ng/ml | 31.9 | 78% | 76% | 11 |
| | | | | | (64%-90%) | (64%-93%) | (3.6-65) |
| C-X-C motif chemokine 2 | P19875 | U | pg/mL | 39 | 68% | 66% | 4 |
| | | | | | (52%-84%) | (52%-83%) | (1.5-14) |
| C-X-C motif chemokine 9 | Q07325 | U | pg/ml | 1120 | 64% | 76% | 5.6 |
| | | | | | (47%-80%) | (47%-92%) | (1.8-24) |
| Dickkopf-related protein 1 | O94907 | U | ng/ml | 0.933 | 62% | 79% | 6.3 |
| | | | | | (44%-80%) | (44%-93%) | (2-27) |
| Elafin | P19957 | U | ng/ml | 8.27 | 68% | 58% | 3 |
| | | | | | (48%-86%) | (48%-86%) | (0.58-16) |
| Fatty acid-binding protein, | P15090 | P | ng/mL | 157 | 66% | 64% | 3.4 |
| adipocyte | | | | | (49%-83%) | (49%-83%) | (1.2-13) |
| Follistatin-related protein 3 | O95633 | P | ng/mL | 64.1 | 71% | 67% | 4.9 |
| | | | | | (54%-88%) | (54%-86%) | (1.7-24) |
| Hepatocyte growth factor-like | P26927 | U | ng/ml | 0.385 | 76% | 72% | 8.3 |
| protein | | | | | (60%-90%) | (60%-89%) | (2.8-37) |
| Insulin-like growth factor- | P18065 | U | ng/mL | 146 | 71% | 63% | 4.1 |
| binding protein 2 | | | | | (54%-86%) | (54%-85%) | (1.2-19) |
| Insulin-like growth factor- | P22692 | U | ng/mL | 318 | 57% | 68% | 2.8 |
| binding protein 4 | | | | | (38%-75%) | (38%-87%) | (0.88-11) |
| Insulin-like growth factor- | Q16270 | U | ng/ml | 594 | 64% | 55% | 2.2 |
| binding protein 7 | | | | | (45%-82%) | (45%-74%) | (0.72-7.7) |
| Insulin-like growth factor- | Q16270 | P | ng/ml | 594 | 64% | 55% | 2.2 |
| binding protein 7 | | | | | (45%-82%) | (45%-74%) | (0.72-7.7) |
| Kallikrein-14 | Q9P0G3 | U | pg/mL | 22.4 | 70% | 90% | 21 |
| | | | | | (53%-90%) | (53%-100%) | (4.4-Inf) |
| Metalloproteinase inhibitor 1 | P01033 | U | ng/mL | 20.8 | 72% | 66% | 4.9 |
| | | | | | (58%-84%) | (58%-84%) | (1.7-18) |
| Metalloproteinase inhibitor 2 | P16035 | U | ng/mL | 23.6 | 74% | 62% | 4.7 |
| | | | | | (58%-89%) | (58%-80%) | (1.6-20) |
| Metalloproteinase inhibitor 4 | Q99727 | U | pg/ml | 33.3 | 78% | 62% | 5.8 |
| | | | | | (65%-89%) | (65%-83%) | (2.1-24) |
| Neutrophil gelatinase- | P80188 | U | ng/mL | 703 | 64% | 69% | 4 |
| associated lipocalin | | | | | (47%-80%) | (47%-86%) | (1.4-13) |
| Neutrophil gelatinase- | P80188 | P | ng/mL | 703 | 64% | 69% | 4 |
| associated lipocalin | | | | | (47%-80%) | (47%-86%) | (1.4-13) |
| Nidogen-1 | P14543 | U | ng/ml | 136 | 66% | 69% | 4.3 |
| | | | | | (50%-82%) | (50%-88%) | (1.5-18) |
| OX-2 membrane glycoprotein | P41217 | U | pg/mL | 0.0142 | 74% | 76% | 8.9 |
| | | | | | (60%-87%) | (60%-92%) | (3.2-40) |
| Pro-interleukin-16 | Q14005 | U | pg/ml | 116 | 64% | 76% | 5.6 |
| | | | | | (47%-80%) | (47%-94%) | (1.8-32) |
| Prolactin | P01236 | P | ng/mL | 26.8 | 71% | 67% | 4.9 |
| | | | | | (55%-86%) | (55%-84%) | (1.8-18) |
| Renin | P00797 | U | ng/mL | 13.3 | 73% | 72% | 7.3 |
| | | | | | (60%-87%) | (60%-88%) | (2.8-27) |
| Tissue factor pathway inhibitor | P10646 | U | pg/ml | 618 | 64% | 75% | 5.4 |
| | | | | | (44%-84%) | (44%-100%) | (1.7-Inf) |
| Tumor necrosis factor | O14763 | P | pg/ml | 418 | 51% | 67% | 2.1 |
| receptor superfamily member 10B | | | | | (31%-71%) | (31%-89%) | (0.6-9.5) |
| Tumor necrosis factor | Q9Y5U5 | U | pg/mL | 0.0101 | 54% | 76% | 3.7 |
| receptor superfamily member 18 | | | | | (40%-68%) | (40%-88%) | (1.7-10) |
| Tumor necrosis factor | O95407 | P | pg/ml | 376 | 75% | 63% | 4.9 |
| receptor superfamily member 6B | | | | | (58%-89%) | (58%-80%) | (1.8-19) |
| WNT1-inducible-signaling | O95388 | U | pg/mL | 8.02 | 67% | 72% | 5.4 |
| pathway protein 1 | | | | | (53%-82%) | (53%-89%) | (1.9-21) |

TABLE 3d
Sensitivity, specificity and odds ratio determined by a threshold at the 75th percentile of the range of
analyte concentrations. RRT treatment discontinuation, if any, starts within 3 days of sample collection.

| | Swiss-Prot | Urine or | | | Sensitivity | Specificity | Odds Ratio |
|---|---|---|---|---|---|---|---|
| Preferred Name | Entry Number | Plasma | Unit | Threshold | (95% CI) | (95% CI) | (95% CI) |
| Antileukoproteinase | P03973 | U | ng/ml | 460 | 43% | 92% | 8.2 |
| | | | | | (23%-67%) | (79%-100%) | (2.4-Inf) |
| Cathepsin B | P07858 | U | ng/ml | 9.58 | 47% | 93% | 12 |
| | | | | | (30%-64%) | (81%-100%) | (3-Inf) |
| C-C motif chemokine 1 | P22362 | U | pg/ml | 8.39 | 36% | 90% | 4.9 |
| | | | | | (19%-54%) | (77%-100%) | (1.4-Inf) |
| C-C motif chemokine 14 | Q16627 | U | ng/mL | 13.8 | 53% | 93% | 15 |
| | | | | | (36%-70%) | (82%-100%) | (4.2-Inf) |
| C-C motif chemokine 16 | O15467 | U | pg/mL | 51.6 | 40% | 93% | 8.5 |
| | | | | | (22%-57%) | (81%-100%) | (2.2-Inf) |
| C-C motif chemokine 23 | P55773 | U | pg/mL | 90.8 | 44% | 97% | 22 |
| | | | | | (27%-61%) | (88%-100%) | (4.8-Inf) |
| C-C motif chemokine 24 | O00175 | U | pg/ml | 152 | 38% | 93% | 8.3 |
| | | | | | (23%-55%) | (81%-100%) | (2.1-Inf) |
| C-C motif chemokine 28 | Q9NRJ3 | U | pg/mL | 79.2 | 50% | 83% | 5 |
| | | | | | (29%-71%) | (56%-100%) | (0.84-Inf) |
| Chitinase-3-like protein 1 | P36222 | U | ng/ml | 109 | 38% | 90% | 5.3 |
| | | | | | (21%-56%) | (76%-100%) | (1.5-Inf) |
| C-X-C motif chemokine 2 | P19875 | U | pg/mL | 135 | 34% | 93% | 7 |
| | | | | | (18%-51%) | (83%-100%) | (1.8-Inf) |
| C-X-C motif chemokine 9 | Q07325 | U | pg/ml | 8460 | 38% | 93% | 8.3 |
| | | | | | (21%-56%) | (82%-100%) | (2.1-Inf) |
| Dickkopf-related protein 1 | O94907 | U | ng/ml | 1.82 | 44% | 93% | 11 |
| | | | | | (28%-61%) | (82%-100%) | (2.9-Inf) |
| Elafin | P19957 | U | ng/ml | 21 | 46% | 92% | 9.5 |
| | | | | | (24%-69%) | (70%-100%) | (1.7-Inf) |
| Fatty acid-binding protein, | P15090 | P | ng/mL | 275 | 37% | 91% | 5.9 |
| adipocyte | | | | | (22%-54%) | (79%-100%) | (1.9-Inf) |
| Follistatin-related protein 3 | O95633 | P | ng/mL | 92.1 | 49% | 88% | 7 |
| | | | | | (31%-68%) | (74%-100%) | (2.1-Inf) |
| Hepatocyte growth | P26927 | U | ng/ml | 2.05 | 42% | 97% | 20 |
| factor-like protein | | | | | (25%-60%) | (88%-100%) | (4.3-Inf) |
| Insulin-like growth | P18065 | U | ng/mL | 628 | 31% | 96% | 12 |
| factor-binding protein 2 | | | | | (16%-48%) | (88%-100%) | (2.5-Inf) |
| Insulin-like growth | P22692 | U | ng/mL | 887 | 37% | 93% | 7.5 |
| factor-binding protein 4 | | | | | (21%-53%) | (81%-100%) | (2-Inf) |
| Insulin-like growth | Q16270 | U | ng/ml | 1020 | 40% | 69% | 1.5 |
| factor-binding protein 7 | | | | | (25%-58%) | (48%-86%) | (0.49-5) |
| Insulin-like growth | Q16270 | P | ng/ml | 1020 | 40% | 69% | 1.5 |
| factor-binding protein 7 | | | | | (25%-58%) | (48%-86%) | (0.49-5) |
| Kallikrein-14 | Q9P0G3 | U | pg/mL | 50.8 | 26% | 90% | 3.2 |
| | | | | | (7%-50%) | (70%-100%) | (0.39-Inf) |
| Metalloproteinase inhibitor 1 | P01033 | U | ng/mL | 47.6 | 44% | 93% | 11 |
| | | | | | (28%-61%) | (83%-100%) | (3.1-Inf) |
| Metalloproteinase inhibitor 2 | P16035 | U | ng/mL | 55 | 40% | 79% | 2.6 |
| | | | | | (25%-58%) | (62%-94%) | (0.84-13) |
| Metalloproteinase inhibitor 4 | Q99727 | U | pg/ml | 90.5 | 40% | 97% | 19 |
| | | | | | (24%-58%) | (88%-100%) | (3.9-Inf) |
| Neutrophil gelatinase- | P80188 | U | ng/mL | 1170 | 34% | 90% | 4.5 |
| associated lipocalin | | | | | (21%-48%) | (77%-100%) | (1.4-Inf) |
| Neutrophil gelatinase- | P80188 | P | ng/mL | 1170 | 34% | 90% | 4.5 |
| associated lipocalin | | | | | (21%-48%) | (77%-100%) | (1.4-Inf) |
| Nidogen-1 | P14543 | U | ng/ml | 273 | 24% | 97% | 8.8 |
| | | | | | (11%-38%) | (88%-100%) | (1.8-Inf) |
| OX-2 membrane | P41217 | U | pg/mL | 49.7 | 24% | 93% | 4.3 |
| glycoprotein | | | | | (11%-38%) | (82%-100%) | (1-Inf) |
| Pro-interleukin-16 | Q14005 | U | pg/ml | 412 | 40% | 90% | 5.8 |
| | | | | | (23%-58%) | (77%-100%) | (1.7-Inf) |
| Prolactin | P01236 | P | ng/mL | 46.7 | 36% | 91% | 5.5 |
| | | | | | (22%-50%) | (79%-100%) | (1.9-Inf) |
| Renin | P00797 | U | ng/mL | 34.7 | 31% | 90% | 3.8 |
| | | | | | (16%-48%) | (77%-100%) | (1.1-Inf) |
| Tissue factor pathway inhibitor | P10646 | U | pg/ml | 1340 | 50% | 92% | 11 |
| | | | | | (29%-72%) | (78%-100%) | (3.1-Inf) |
| Tumor necrosis factor | O14763 | P | pg/ml | 630 | 33% | 100% | >8 |
| receptor superfamily member 10B | | | | | (15%-52%) | (100%-100%) | |
| Tumor necrosis factor | Q9Y5U5 | U | pg/mL | 1.53 | 35% | 93% | 7.4 |
| receptor superfamily member 18 | | | | | (22%-50%) | (83%-100%) | (2.5-Inf) |
| Tumor necrosis factor | O95407 | P | pg/ml | 691 | 61% | 88% | 11 |
| receptor superfamily member 6B | | | | | (44%-77%) | (73%-100%) | (3.5-Inf) |
| WNT1-inducible-signaling | O95388 | U | pg/mL | 16.6 | 24% | 90% | 2.8 |
| pathway protein 1 | | | | | (12%-39%) | (78%-100%) | (0.78-Inf) |

While the invention has been described and exemplified in sufficient detail for those skilled in this art to make and use it, various alternatives, modifications, and improvements should be apparent without departing from the spirit and scope of the invention. The examples provided herein are representative of preferred aspects, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred aspects and optional features, modification and variation of the concepts herein disclosed may be resorted to by those skilled in the art, and that such modifications and variations are considered to be within the scope of this invention as defined by the appended claims.

Other aspects are set forth within the following claims.

1. A method for assessing the likelihood that a subject will benefit from treatment with continued renal replacement therapy (RRT), comprising:
detecting a level of one or more biomarkers, wherein each of the one or more biomarkers are independently selected from the list consisting of C-C-motif chemokine and, kallikrein-14, antileukoproteinase, cathepsin B, C-C motif chemokine 1, C-C motif chemokine 16, C-C motif chemokine 23, C-C motif chemokine 24, C-C motif chemokine 28, chitinase-3-like protein 1, C-X-C motif chemokine 2, C-X-C motif chemokine 9, dickkopf-related protein 1, elafin, fatty acid-binding protein adipocyte, follistatin-related protein 3, hepatocyte growth factor-like protein, insulin-like growth factor-binding protein 2, insulin-like growth factor binding protein 4, insulin-like growth factor binding protein 7, metalloproteinase inhibitor 1, metalloproteinase inhibitor 2, metalloproteinase inhibitor 4, neutrophil gelatinase-associated lipocalin, nidogen-1, OX-2 membrane glycoprotein, pro-interleukin-16, prolactin, renin, tissue factor pathway inhibitor, tumor necrosis factor receptor superfamily member 10B, tumor necrosis factor receptor superfamily member 18, tumor necrosis factor receptor superfamily member 6B, and WNT1-inducible signaling pathway protein 1 in at least one body fluid sample obtained from the subject to produce one or more assay results produced by an analyte binding assay;
correlating the one or more assay results to a likelihood that the subject will benefit from continued RRT or a likelihood that the subject will not benefit from continued RRT,
administering continued RRT if the subject is at an increased likelihood from benefiting from continued RRT; and
discontinuing RRT if the subject is not at an increased likelihood of benefiting from continued RRT.

2-4. (canceled)

5. The method of claim 1, wherein the RRT comprises continuous renal replacement therapy, intermittent renal replacement therapy, prolonged intermittent renal replacement therapy, continuous hemodialysis, continuous hemofiltration, continuous hemodiafiltration, intermittent hemodialysis, intermittent hemofiltration, intermittent hemodiafiltration, acute hemodialysis, peritoneal dialysis, slow continuous ultrafiltration, or sustained low efficiency dialysis.

6. (canceled)

7. The method of claim 1, wherein the correlation comprises assigning the subject to a predetermined subpopulation of individuals exhibiting a known status with regard to benefiting from administration of continued RRT, wherein assigning comprises comparing an assay result to a threshold selected in a population study, wherein the threshold separates the population into a first subpopulation having measurements above the threshold which has an increased predisposition for benefiting from treatment with continued RRT relative to a second subpopulation having measurements at or below the threshold.

8. The method of claim 1, wherein the correlation comprises assigning the subject to a predetermined subpopulation of individuals exhibiting a known status for having or for not having successfully discontinued RRT, wherein assigning comprises comparing an assay result to a threshold selected in a population study, wherein the threshold separates the population into a first subpopulation having measurements above the threshold which has not successfully discontinued RRT and a second population having measurements at or below the threshold which has successfully discontinued RRT.

9. The method of claim 1, wherein the correlation comprises comparing an assay result to a baseline previously measured in the subject.

10. The method of claim 7, wherein the subject is assigned to the first subpopulation, or wherein the subject is assigned to the second subpopulation.

11. The method of claim 9, wherein the assay result is higher than the baseline.

12. (canceled)

13. The method of claim 1, wherein the RRT is administered for more than 1, more than 2, or more than 3 days after the sample is obtained.

14. (canceled)

15. The method of claim 9, wherein the assay result is not higher than the baseline.

16-18. (canceled)

19. The method of claim 1, wherein the analyte binding assay comprises an antibody.

20. The method of claim 1, wherein the at least one body fluid sample comprises a urine sample.

21. The method of claim 1, wherein the at least one body fluid sample comprises a whole blood, plasma sample, or serum sample.

22. The method of claim 1, wherein the at least one body fluid sample comprises a urine sample and a plasma sample.

23. The method of claim 1, wherein the subject has one or more of congestive heart failure, diabetes mellitus, hypertension, coronary artery disease, proteinuria, cirrhosis, chronic kidney disease, cancer, chronic obstructive pulmonary disease, anemia, sepsis, shock, or hypotension at the time the sample is obtained.

24. The method of claim 23, wherein the subject experienced surgery or trauma within about 12, 24, 36, 48, 72, 96, or 120 hours prior to the time the sample is obtained.

25. The method of claim 1, wherein the at least one body fluid sample is collected within about 6, 8, 12, 24, 36, 48, or 72 hours of RRT having been initiated.

26. The method of claim 25, wherein RRT had been initiated within about 6, 8, 12, 24, 36, 48, or 72 hours of the subject meeting the criteria for KDIGO stage 2 acute kidney injury or within about 6, 8, 12, 24, 36, 48, or 72 hours of the subject meeting the criteria for KDIGO stage 3 acute kidney injury.

27. The method of claim 1, wherein the subject has acute kidney injury (AKI) at the time the sample is obtained.

28. The method of claim 27, wherein the subject is in KDIGO stage 2 or 3 of acute kidney injury (AKI) at the time the sample was obtained.

29-45. (canceled)

46. A kit comprising one or more binding reagents, each of which binds one of the one or more biomarkers of claim 1, wherein one of the one or more binding reagents binds C-C motif chemokine 14.

47-124. (canceled)
